SEQUENCE LISTING (18 sequences)

SEQ ID NO: 1 - protein, 13 aa, Escherichia coli
CGNSSRTITGDTC

SEQ ID NO: 2 - protein, 6 aa, Artificial Sequence
  CTA fusion peptide
APRPGP

SEQ ID NO: 3 - protein, 16 aa, Artificial Sequence
  CTA fusion peptide
ASRCAELCCNPACPAP

SEQ ID NO: 4 - protein, 23 aa, Artificial Sequence
  CTA fusion peptide
ANSSNYCCELCCNPACTGCYPGP

SEQ ID NO: 5 - DNA, 1148 bp, Escherichia coli
atgaaaaatataactttcattttttttattttattagcatcgccattatatgcaaatggcgacaaattataccgtgctgactctagacccccagatgaaataaaacgttccggaggtcttatgcccagagggcataatgagtacttcgatagaggaactcaaatgaatattaatctttatgatcacgcgagaggaacacaaaccggctttgtcagatatgatgacggatatgtttccacttctcttagtttgagaagtgctcacttagcaggacagtctatattatcaggatattccacttactatatatatgttatagcgacagcaccaaatatgtttaatgttaatgatgtattaggcgtatacagccctcacccatatgaacaggaggtttctgcgttaggtggaataccatattctcagatatatggatggtatcgtgttaattttggtgtgattgatgaacgattacatcgtaacagggaatatagagaccggtattacagaaatctgaatatagctccggcagaggatggttacagattagcaggtttcccaccggatcaccaagcttggagagaagaaccctggattcatcatgcaccacaaggttgtggagattcatcaagaacaattacaggtgatacttgtaatgaggagacccagaatctgagcacaatatatctcaggaaatatcaatcaaaagttaagaggcagatattttcagactatcagtcagaggttgacatatataacagaattcggaatgaattatgaataaagtaaaatgttatgttttatttacggcgttactatcctctctatgtgcatacggagctccccagtctattacagaactatgttcggaatatcgcaacacacaaatatatacgataaatgacaagatactatcatatacggaatcgatggcaggcaaaagagaaatggttatcattacatttaagagcggcgcaacatttcaggtcgaagtcccgggcagtcaacatatagactcccaaaaaaaagccattgaaaggatgaaggacacattaagaatcacatatctgaccgagaccaaaattgataaattatgtgtatggaataataaaacccccaattcaattgcggcaatcagtatggaaaactag

SEQ ID NO: 6 - protein, 258 aa, Escherichia coli
MKNITFIFFILLASPLYANGDKLYRADSRPPDEIKRSGGLMPRGHNEYFDRGTQMNINLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGDSSRTITGDTCNEETQNLSTIYLRKYQSKVKRQIFSDYQSEVDIYNRIRNEL

SEQ ID NO: 7 - DNA, 723 bp, Escherichia coli
aatggcgacaaattataccgtgctgactctagacccccagatgaaataaaacgttccggaggtcttatgcccagagggcataatgagtacttcgatagaggaactcaaatgaatattaatctttatgatcacgcgagaggaacacaaaccggctttgtcagatatgatgacggatatgtttccacttctcttagtttgagaagtgctcacttagcaggacagtctatattatcaggatattccacttactatatatatgttatagcgacagcaccaaatatgtttaatgttaatgatgtattaggcgtatacagccctcacccatatgaacaggaggtttctgcgttaggtggaataccatattctcagatatatggatggtatcgtgttaattttggtgtgattgatgaacgattacatcgtaacagggaatatagagaccggtattacagaaatctgaatatagctccggcagaggatggttacagattagcaggtttcccaccggatcaccaagcttggagagaagaaccctggattcatcatgcaccacaaggttgtggagattcatcaagaacaattacaggtgatacttgtaatgaggagacccagaatctgagcacaatatatctcaggaaatatcaatcaaaagttaagaggcagatattttcagactatcagtcagaggttgacatatataacagaattcggaatgaattatga

SEQ ID NO: 8 - protein, 240 aa, Escherichia coli
NGDKLYRADSRPPDEIKRSGGLMPRGHNEYFDRGTQMNINLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGDSSRTITGDTCNEETQNLSTIYLRKYQSKVKRQIFSDYQSEVDIYNRIRNEL

SEQ ID NO: 9 - DNA, 312 bp, Escherichia coli
gctccccagtctattacagaactatgttcggaatatcgcaacacacaaatatatacgataaatgacaagatactatcatatacggaatcgatggcaggcaaaagagaaatggttatcattacatttaagagcggcgcaacatttcaggtcgaagtcccgggcagtcaacatatagactcccaaaaaaaagccattgaaaggatgaaggacacattaagaatcacatatctgaccgagaccaaaattgataaattatgtgtatggaataataaaacccccaattcaattgcggcaatcagtatggaaaactag

SEQ ID NO: 10 - protein, 124 aa, Escherichia coli
MNKVKCYVLFTALLSSLCAYGAPQSITELCSEYRNTQIYTINDKILSYTESMAGKREMVIITFKSGATFQVEVPGSQHIDSQKKAIERMKDTLRITYLTETKIDKLCVWNNKTPNSIAAISMEN

SEQ ID NO: 11 - DNA, 312 bp, Artificial Sequence
  Nucleotide sequence of LT B G33D
gctccccagtctattacagaactatgttcggaatatcgcaacacacaaatatatacgataaatgacaagatactatcatatacggaatcgatggcagacaaaagagaaatggttatcattacatttaagagcggcgcaacatttcaggtcgaagtcccgggcagtcaacatatagactcccaaaaaaaagccattgaaaggatgaaggacacattaagaatcacatatctgaccgagaccaaaattgataaattatgtgtatggaataataaaacccccaattcaattgcggcaatcagtatggaaaactag

SEQ ID NO: 12 - protein, 103 aa, Artificial Sequence
  Amino acid sequence of LT B G33D
APQSITELCSEYRNTQIYTINDKILSYTESMADKREMVIITFKSGATFQVEVPGSQHIDSQKKAIERMKDTLRITYLTETKIDKLCVWNNKTPNSIAAISMEN

SEQ ID NO: 13 - protein, 240 aa, Artificial Sequence
  Amino acid sequence of LT-K63 mutant
NGDKLYRADSRPPDEIKRSGGLMPRGHNEYFDRGTQMNINLYDHARGTQTGFVRYDDGYVSTKLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGDSSRTITGDTCNEETQNLSTIYLRKYQSKVKRQIFSDYQSEVDIYNRIRNEL

SEQ ID NO: 14 - protein, 240 aa, Artificial Sequence
  Amino acid sequence of LT-R72
NGDKLYRADSRPPDEIKRSGGLMPRGHNEYFDRGTQMNINLYDHARGTQTGFVRYDDGYVSTSLSLRSAHLRGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGDSSRTITGDTCNEETQNLSTIYLRKYQSKVKRQIFSDYQSEVDIYNRIRNEL

SEQ ID NO: 15 - protein, 240 aa, Artificial Sequence
  Amino acid sequence of LT-R192G
NGDKLYRADSRPPDEIKRSGGLMPRGHNEYFDRGTQMNINLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGDSSGTITGDTCNEETQNLSTIYLRKYQSKVKRQIFSDYQSEVDIYNRIRNEL

SEQ ID NO: 16 - DNA, 1369 bp, Vibrio cholerae
actaaatagtatattttgatttttgatttttgatttcaaataatacaaatttatttacttatttaattgttttgatcaattatttttctgttaaacaaagggagcatatatggtaaagataatatttgtgttttttattttcttatcatcattttcatatgcaaatgatgataagttatatcgggcagattctagacctcctgatgaaataaagcagtcaggtggtcttatgccaagaggacagagtgagtactttgaccgaggtactcaaatgaatatcaacctttatgatcatgcaagaggaactcagacgggatttgttaggcacgatgatggatatgtttccacctcaattagtttgagaagtgcccacttagtgggtcaaactatattgtctggtcattctacttattatatatatgttatagccactgcacccaacatgtttaacgttaatgatgtattaggggcatacagtcctcatccagatgaacaagaagtttctgctttaggtgggattccatactcccaaatatatggatggtatcgagttcattttggggtgcttgatgaacaattacatcgtaataggggctacagagatagatattacagtaacttagatattgctccagcagcagatggttatggattggcaggtttccctccggagcatagagcttggagggaagagccgtggattcatcatgcaccgccgggttgtgggaatgctccaagatcatcgatgagtaatacttgcgatgaaaaaacccaaagtctaggtgtaaaattccttgacgaataccaatctaaagttaaaagacaaatattttcaggctatcaatctgatattgatacacataatagaattaaggatgaattatgattaaattaaaatttggtgttttttttacagttttactatcttcagcatatgcacatggaacacctcaaaatattactgatttgtgtgcagaataccacaacacacaaatatatacgctaaatgataagatattttcgtatacagaatctctagctggaaaaagagagatggctatcattacttttaagaatggtgcaatttttcaagtagaagtaccaggtagtcaacatatagattcacaaaaaaaagcgattgaaaggatgaaggataccctgaggattgcatatcttactgaagctaaagtcgaaaagttatgtgtatggaataataaaacgcctcatgcgattgccgcaattagtatggcaaattaagatataaaaaagcccacctcagtgggcttttttgtggttcgatgatgagaagcaaccgttttgcccaaacatgtattactgcaagtatgatgtttttattccacatccttag

SEQ ID NO: 17 - protein, 258 aa, Vibrio cholerae
MVKIIFVFFIFLSSFSYANDDKLYRADSRPPDEIKQSGGLMPRGQSEYFDRGTQMNINLYDHARGTQTGFVRHDDGYVSTSISLRSAHLVGQTILSGHSTYYIYVIATAPNMFNVNDVLGAYSPHPDEQEVSALGGIPYSQIYGWYRVHFGVLDEQLHRNRGYRDRYYSNLDIAPAADGYGLAGFPPEHRAWREEPWIHHAPPGCGNAPRSSMSNTCDEKTQSLGVKFLDEYQSKVKRQIFSGYQSDIDTHNRIKDEL

SEQ ID NO: 18 - protein, 124 aa, Vibrio cholerae
MIKLKFGVFFTVLLSSAYAHGTPQNITDLCAEYHNTQIYTLNDKIFSYTESLAGKREMAIITFKNGAIFQVEVPGSQHIDSQKKAIERMKDTLRIAYLTEAKVEKLCVWNNKTPHAIAAISMAN

(19) United States
(12) Patent Application Publication
(10) Pub. No.: US 2006/0002960 A1
(43) Pub. Date: Jan. 5, 2006

(54) GM1 binding deficient exotoxins for use as immunoadjuvants

(76) Inventors: Paul Zoeteweij, Gaithersburg, MD (US); Gregory M. Glenn, Gaithersburg, MD (US); Larry R. Ellingsworth, Gaithersburg, MD (US)

Correspondence Address: MORGAN LEWIS & BOCKIUS LLP, 1111 PENNSYLVANIA AVENUE NW, WASHINGTON, DC, 20004, US

(21) Appl. No.: 11/007,282

(22) Filed: Dec. 9, 2004

Related U.S. Application Data
(60) Provisional application No. 60/527,751, filed on Dec. 9, 2003.
(60) Provisional application No. 60/579,288, filed on Jun. 15, 2004.
(60) Provisional application No. 60/613,520, filed on Sep. 28, 2004.

(51) Int. Cl. A61K 39/02

(52) U.S. Cl. 424/235.1

(57) ABSTRACT

Addition of a bacterial ADP-ribosylating exotoxin (bARE) to a formulation (e.g., immunogen or vaccine) or a system (e.g., patch or kit) for immunization enhances the immune response in a subject to one or more components of the formulation. Binding of the B subunit of a bARE to ganglioside GM1 of the subject in vivo, however, mediates toxicity and limits the use of native bARE as adjuvants. Mutation or in vitro coupling of the B subunit to ligands such as GM1 inhibits binding to GM1 in vivo, thereby eliminating toxicity but retaining desired adjuvant activity. The use of such detoxified, GM-1 binding deficient exotoxins provides a safe and potent new strategy for development of effective formulation for immunization.

### CROSS-REFERENCE TO RELATED APPLICATIONS

This application claims priority to provisional U.S. Patent Appl. No. 60/527,751, filed Dec. 9, 2003, to provisional U.S. Patent Appl. No. 60/579,288, filed Jun. 15, 2004, and to provisional U.S. Patent Appl. No. 60/613,520, filed Sep. 28, 2004, all of which are incorporated by reference herein in their entirety.

### FIELD OF THE INVENTION

The invention is in the field of immunoadjuvants. The invention relates to a formulation comprising a bacterial ADP-ribosylating exotoxin modified by mutation of a B subunit and/or in vitro coupling and/or binding of the B subunit with its cognate receptor, a binding portion thereof or any other chemical ligand, to inhibit subsequent binding to complex gangliosides in vivo, for use as an in vivo immunoadjuvant with reduced toxicity.

### BACKGROUND OF THE INVENTION

Exotoxins such as, for example, cholera toxin (CT), Escherichia coli heat-labile enterotoxin (LT), diptheria toxin (DT), pertussis toxin (PT), and Pseudomonas aeruginosa exotoxin A (ETA) are bacterial products that enhance the immune response. After administration by an oral, intramuscular, buccal, nasal, rectal, pulmonary, intradermal, subcutaneous, or epicutaneous route, a bacterial exotoxin can induce a regional and/or systemic immune response to itself as well as to a coadministered antigen (Elson & Dertzbaugh, 1994; Tomasi et al., 1997; Enioutina et al., 2000; Glenn et al., 2000; Scharton-Kersten et al., 2000).

Bacterial ADP-ribosylating exotoxins (bARE) are organized as A:B heterodimers consisting of one A and five B subunits (AB₅). Studies using exotoxins in which the A or B subunit is deleted show that both A and B subunits have adjuvant activities (De Haan et al., 1998; De Haan et al., 1999; Lian et al., 2000; Scharton-Kersten et al., 2000), although not always as good as intact heterodimers. Application of exotoxins in immunizations has been described, but is limited in humans due to severe toxicity such as inflammation and diarrhea (Clemens et al., 1988; van Ginkel et al., 2000; U.S. Pat. No. 6,576,244). To exert its toxic action, binding of the B subunit to the ganglioside GM1 on the cell surface is followed by the A subunit entering the cell. Then, the intracellular ADP-ribosylating activity of the A subunit in intestinal epithelia leads to fluid loss and diarrhea (Krueger & Barbieri, 1995; Sears & Kaper, 1996). To reduce toxicity, modified exotoxins are described in which the A subunit is mutated or deleted to reduce or eliminate ribosylation activity, respectively (WO03/047619; U.S. Pat. Nos. 6,436,407 and 6,576,244; De Haan et al., 1998; De Haan et al., 1999). However, these formulations may retain toxicity due to residual ribosylation activity or contamination with trace amounts of intact toxin. Or, there is an undesirable loss of adjuvanticity (Lycke et al., 1992; Tamura et al., 1994).

Taken together, it is believed that an intact A subunit with ribosylating activity non-covalently complexed with a GM 1-binding pentameric B subunit is optimal for adjuvant activity. Exotoxins with B subunit modifications that interfere with cellular binding are generally not considered potent adjuvants. Using oral routes of vaccine delivery in which adverse effects are easily assessed, attempts to reduce toxicity by mutating the B subunit lead to undesired decrease or change in adjuvant activity (Guidry et al., 1997; Aman et al, 2001). Only in some studies using intranasal administration do B subunit mutations show adjuvant activity (De Haan et al., 1998; De Haan et al., 1999). However, the atypical nasal mucosal environment has been associated with specific translocation of exotoxin (or exotoxin subunits) and co-administered antigens to the central nervous system raising concerns about undesirable toxicity which is difficult to predict in current available animal models (van Ginkel et al., 2000; Couch, 2004).

Adjuvant activity of a bARE modified by coupling to B subunit binding ligands has not been described. Beignon et al. (2001) recently reported that LT complexed to GM1 in vitro before in vivo administration for transcutaneous immunization (TCI) was a poor immunogen. Based on this observation, the complex would also be assumed to have no adjuvant activity. In addition, an LT mutant with a point mutation in B subunit to prevent GM1 binding failed to elicit potent immune responses (Guidry et al., 1997). This supported the assumption that interference with in vivo GM1 binding is deleterious for vaccine development purposes (Tomasi et al., 1997).

### SUMMARY OF THE INVENTION

The present invention is directed to the use of ganglioside-binding deficient exotoxins that address the problem of reducing toxicity while retaining adjuvant activity. Further advantages and improvements are discussed below or would be apparent from the disclosure herein. The modes of administration are explicitly set forth and do not include intranasal administration.

### BRIEF DESCRIPTION OF THE DRAWINGS

FIGS. 1A-B. Transcutaneous immunization (TCI) with liquid formulated, LT/GM-1 adjuvanted tetanous toxoid. C57B1/6 mice were prepared by shaving dorsal caudal surface one day before immunization. The shaven skin was tape stripped (10×) and the skin hydrated with saline immediately before immunization. Groups of 5 mice were immunized by application of a 25 μl volume containing 10 Lf TT alone, TT admixed with 10 μg LT, or TT admixed with LT complexed with GM-1 (10 μg, 15 μg or 20 μg). The solution was applied for 1 hour and rinsed with warm water to remove excess vaccine. All groups were immunized 3 times (day 1, 15, 29) and serum collected 2 weeks after the third immunization. Serum anti-TT IgG (panel A) and anti-LT IgG titers were determined by an ELISA method. Antibody titers for each animal are reported as ELISA Units (EU), which is the serum dilution equal to 1OD unit at 405 nm. The geometric mean titer for each group is indicated. A T test was used to compare differences between mice immunized with TT alone to those immunized with LT or LT/GM-1 adjuvanted vaccine.

FIG. 2. Topical immunization and adjuvanticity of LT, LTGly33Asp (LTG33D) and LTGM1 liquid formulated patch. C57B1/6 mice were prepared by shaving dorsal caudal one day before immunization. Immediately before immunization, the shaven skin was saline hydrated and pre-treated with emery paper (10 strokes) to disrupt the stratum corneum. Patches were constructed of a 1 cm² gauze patch affixed to an adhesive backing and loaded with 25 μl containing 10 Lf tetanus toxoid (TT) in phosphate buffered saline (no LT) or admixed with wild type (wt) LT (10 μg), or LTGly33Asp (10 and 50 μg) or LT-GM1 (10 and 50 μg). Patches were applied for 1 hour, removed and the skin rinsed with water. Groups of 9-10 mice were immunized with patches on study day 1 and 15 and serum was collected two weeks after the second dose. An ELISA method was used to determine anti-TT IgG titers. Antibody titers are reported as ELISA Units (EU), which is the serum dilution equal to 1.0 OD at 405 nm. The geometric mean titer is indicated for each group. A T test was used to compare antibody titers between groups receiving adjuvanted TT to the group receiving the non-adjuvanted vaccine.

FIG. 3. Transcutaneous immunization with wild type (wt) LT and LTGly33Asp (LTG33D) adjuvanted ovalbumin (OVA). C57B1/6 mice were prepared by shaving dorsal caudal one day before immunization. Immediately before patch application, the shaven skin was saline hydrated and pretreated with emery paper (10 strokes) to disrupt the stratum corneum. Patches were constructed of a 1.0 cm² gauze pad affixed to an adhesive backing. Patches were loaded with 25 μl containing 150 μg OVA alone (no LT) or admixed with 25 μg of wild type (wt) LT or LTGly33Asp. Patches were applied overnight, removed and the skin rinsed with water. All mice were immunized with three doses (day 1, 15 and 29) and serum collected 2 weeks after the third dose. Serum antibodies to OVA were determined by an ELISA method. Serum antibody titers are reported as ELISA Units (EU), which is the serum dilution equal to 1.0 OD unit at 405 nm. The geometric mean titer for groups of 10 mice is indicated.

FIGS. 4A-B. LT and LTGly33Asp (LTG33D) potentiate cellular immune responses topical co-administered ovalbumin (OVA). C57B1/6 mice were prepared by shaving dorsal caudal one day before immunization. Immediately before immunization, the shaven skin was saline hydrated and pre-treated with emery paper (10 strokes) to disrupt the stratum corneum. OVA (150 μg) was admixed with phosphate buffered saline (no adjuvant) or with 25 μg wild type (wt) LT or LTGLY33ASP and 25 μl applied to a 1 cm² gauze patch affixed to an adhesive backing. Patches were applied overnight and the skin rinsed with water. Two weeks after three immunizations (day 1, 15, 29), inguinal lymph nodes (LN) and spleens were collected from groups of 10 mice, pooled, and the lymphocytes re-stimulated in cell culture with OVA or LT. The number of OVA and LT lymphocytes induced to produce IFN-gamma (panel A) and IL-4 (panel B) was determined by ELISPOT analysis.

FIGS. 5A-B. Topical delivery of wild type LT and LTGly33Asp (LT(G33D)) and potentiation of the humoral immune response to parenteral immunization with tetanus toxoid vaccine. C57B1/6 mice were prepared by shaving dorsal caudal one day before immunization. Immediately prior to immunization, the shaven skin was saline hydrated and pre-treated with emery paper (10 strokes) to disrupt the stratum corneum. Tetanus toxoid (0.2 Lf) was intradermal injected into the pretreated skin and a 1 cm² gauze patch affixed to an adhesive backing was loaded with 25 μl of phosphate buffered saline (no adjuvant), or wild type (wt) LT or LT(G33D) at the indicated doses. Patches were applied overnight and the skin rinsed with water. Groups of 8-10 mice were immunized with patches on study day 1 and 15 and serum was collected two weeks after the second dose. An ELISA method was used to determine anti-tetanus toxoid (TT) and anti-LT titers (panels A and B, respectively). Antibody titers are reported as ELISA Units (EU), which is the serum dilution equal to 1.0 OD at 405 nm. The geometric mean titer is indicated for each group.

FIGS. 6A-B. Topical delivery of wild type LT and LTGly33Asp ((LT(G33D)) and potentiation of the humoral immune response to parenteral immunization with ovalbumin. C57B1/6 mice were prepared by shaving dorsal caudal one day before immunization. Immediately before immunization, the shaven skin was saline hydrated and pre-treated with emery paper (10 strokes) to disrupt the stratum corneum. Ovalbumin (150 μg) was intradermal injected into the pretreated skin and a 1 cm² gauze patch affixed to an adhesive backing was loaded with 25 μl of phosphate buffered saline (no adjuvant), or wild type (wt) LT or LT(G33D) at the indicated doses. Patches were applied overnight and the skin rinsed with water. Groups of 5-9 mice were immunized with patches on study day 1 and 15 and serum was collected two weeks after the second dose. An ELISA method was used to determine anti-ovalbumin and anti-LT titers (panels A and B, respectively). Antibody titers are reported as ELISA Units (EU), which is the serum dilution equal to 1.0 OD at 405 nm. The geometric mean titer is indicated for each group.

FIGS. 7A-B. Use of soluble GM1 ganglioside to attenuate LTArg192Gly (LTR192G) reactogenicity without affecting immune stimulating activity to a bystander antigen. Panel A. Groups of C57B1/6 mice (N=7/group) were shaved near the base of the tail and intradermal injected with 0.5 μg wild type LT or LTR192G alone or complexed with soluble GM1 ganglioside at a molar ration of 1:16. Calipers were used to measure the diameter of injection site induration at the indicated time points. Panel B. Mice were prepared for immunization by shaving dorsal caudal one day before immunization. Groups mice were immunized by intradermal injection of 10 Lf tetanus toxoid (TT) alone (in PBS) or admixed with 0.5 μg LTR192G. LTR192G was complexed with 0.5 ng, 12.5 ng or 750 ng of soluble GM1 ganglioside. All groups were immunized on study day 1 and 15 and serum was collected 1 week after the second dose. Serum anti-TT IgG titers were determined by an ELISA method. Antibody titers are reported as ELISA Units (EU), which is the serum dilution equal to 1OD unit at 405 nm. The geometric mean titers for groups of 5 mice is indicated.

FIGS. 8A-B. Effect of skin abrasion on the immune response to ID injected tetanus toxoid. Groups of mice were shaved 1-2 days before immunization. One group was not pre-treated and did not receive a patch. Immediately before immunization, the second group was pretreated by hydration with PBS and emery paper (10 strokes). Both groups were immunized with 0.5 Lf tetanus toxoid by ID injection in the shaven area. Group 2 received a placebo patch applied over the injection site, removed after 18 hr. Serum antibody titers to TT were determined by the ELISA method. Anti-TT titers are reported as ELISA Units (EU), which is the serum dilution equal to 1 OD unit at 405 nm. The geometric mean titer is indicated for each group (N=5/group). Panel A. Two weeks after a single immunization (study day 14). Panel B. Two weeks after the second immunization (study day 28).

FIGS. 9A-B. Effect of skin abrasion on immune response to parenteral injected tetanus toxoid. FIG. 9A. C57B1/6 mice were prepared for immunization by shaving the dorsal caudal surface two days before vaccination. Immediately before immunization, the shaven skin was pretreated by saline hydration and emery paper (10 strokes) to disrupt the stratum corneum (groups 2-5). An intradermal injection of 0.5 Lf of tetanus toxoid was administered and a 1 cm² gauze patch on an adhesive backing was applied over the injection site. Patches were loaded with 25 μl of phosphate buffered saline (placebo) or LT (0.1, 1.0 or 10 μg). Patches were applied overnight (˜18 hr), removed and the skin rinsed with water. A separate group (1) was immunized by intradermal injection with 0.5 Lf tetanus toxoid without skin pretreatment and without a patch. Serum was collected at 2 weeks post immunization and antibody titers to tetanus toxoid determined by an ELISA method. Antibody titers are reported as ELISA Units (EU), which is the serum dilution equal to 1.0 OD at 405 nm. The geometric mean titer (GMT) for groups of 7-8 mice is indicated. FIG. 9B. Student T test was used to determine differences between groups.

FIG. 10. Generation of LT toxin neutralizing antibodies by vaccination with LTGly33Asp. C57B1/6 mice were immunized by intradermal injection with 0.5 μg of the mutant LT or topically with 25 μg of wild type LT on study days, 1, 22 and 43. Serum was collected two weeks after the third immunization. Serum from non-immunized (naïve) and immunized mice was two-fold serially diluted and mixed with a toxic amount (5 ng) of wild type LT. After 30-minutes the mixture was added to Y1 cells (5×10⁵ cells/ml) in 96 well microculture plates. The cells were incubated at 37° C. overnight. The cells were stained by adding 0.05 ml of 0.01% neutral red to the cultures for 2-3 hours. The cultures were then washed with phosphate buffered saline to remove the rounded non-adherent cells. The dye was extracted from the remaining adherent cells with acetic acid and ethanol and the color read at 530 nm with an ELISA plate reader.

FIG. 11. Complete cDNA sequence of LT (SEQ ID NO: 5)

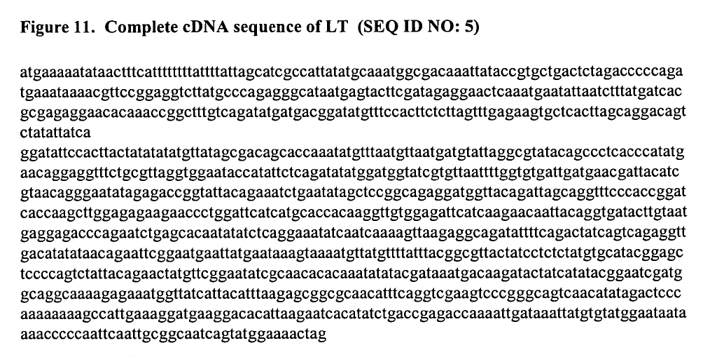

FIG. 12. Wild type (wt) amino acid sequence of LT A from H10407 with signal sequence attached. (SEQ ID NO: 6)

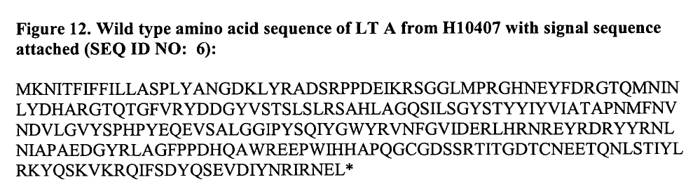

FIG. 13. Wild type LT A nucleotide sequence without signal sequence attached (SEQ ID NO: 7).

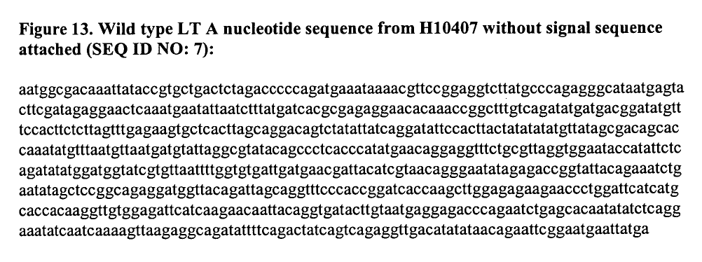

FIG. 14. Wild type LT A amino acid sequence without signal sequence attached (SEQ ID NO: 8).

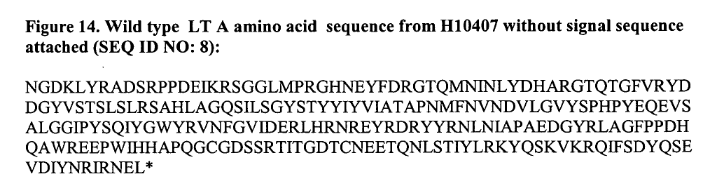

FIG. 15. Wild type nucleotide sequence of LT B (SEQ ID NO: 9).

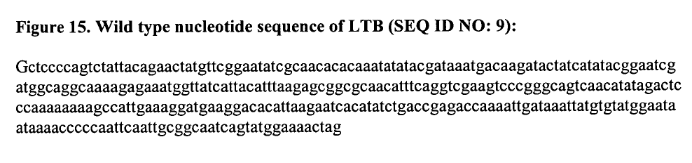

FIG. 16. Wild type amino acid sequence of LT B (SEQ ID NO: 10).

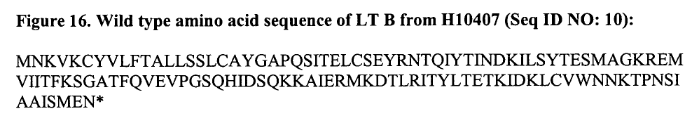

FIG. 17. Nucleotide sequence of LT B G33D (SEQ ID NO: 11). Residue position for the LT and CT mutants is based on the wild type B subunit amino acid sequence without the signal sequence.

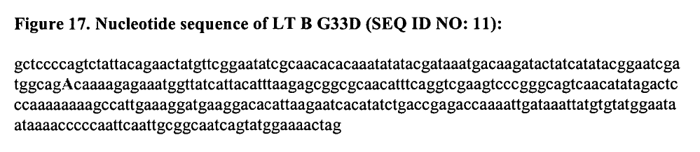

FIG. 18. Amino acid sequence of LT B G33D (SEQ ID NO: 12).

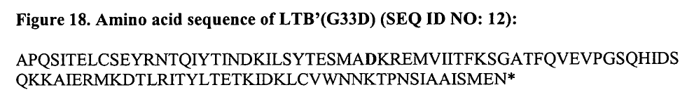

FIG. 19. Amino acid sequence of LT-K63 mutant (SEQ ID NO: 13).

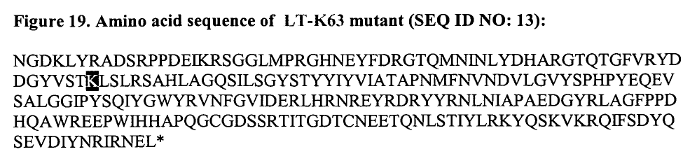

FIG. 20. Amino acid sequence of LT-R72 (SEQ ID NO: 14).

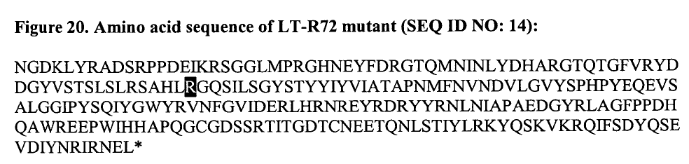

FIG. 21. Amino acid sequence of LT-R192G (SEQ ID NO: 15).

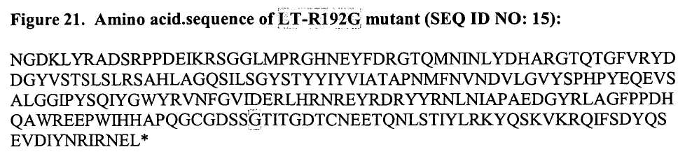

FIG. 22. Nucleotide sequence of CT (SEQ ID NO: 16).

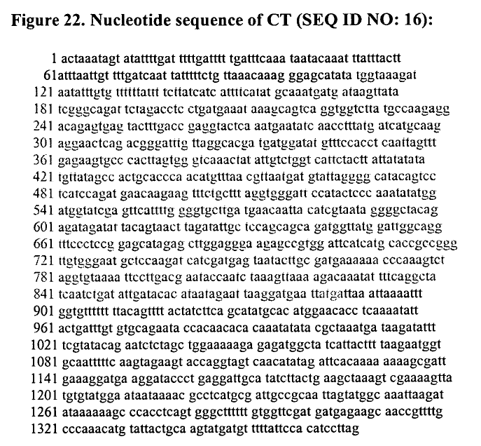

FIG. 23. Amino acid sequence of CT A (SEQ ID NO: 17).

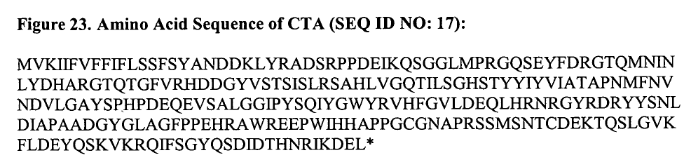

FIG. 24. Amino acid sequence of CT B (SEQ ID NO: 18).

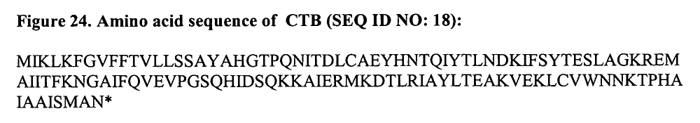

### DETAILED DESCRIPTION OF THE INVENTION

Exotoxins bind to gangliosides on cells. Exotoxins include at least one catalytic subunit generally referred to in the art as the “A subunit” and at least one binding subunit generally referred to in the art as the “B subunit.” A large class of exotoxins naturally bind the GM-1 receptor on cells. A “GM-1 binding deficient exotoxin” as used herein refers to an exotoxin which has been modified such that GM-1 binding is inhibited or reduced in a manner sufficient to reduce the toxicity.

The GM-1 binding deficient exotoxin can be produced by substituting one or more amino acids in at least one B subunit of the exotoxin and/or by coupling at least one B subunit of the exotoxin to a molecule that is effective to inhibit binding of the exotoxin to GM-1. For example, one could use a formulation comprising a bacterial ADP-ribosylating exotoxin modified by mutation of a B subunit and/or in vitro coupling and/or binding of the B subunit with its cognate receptor, a binding portion thereof or any other chemical ligand, to inhibit subsequent binding to complex gangliosides in vivo, for use as an in vivo immunoadjuvant with reduced toxicity. The amino acid substitutions can be introduced as one or more single point mutations in the GM-1 binding pocket. An exotoxin may include one or more subunits having one or more mutations, having one or more in vitro coupled ligands or any binding portion thereof, having one or more bound ligands or any binding portion thereof, having one or more cognate receptors or any binding portion thereof or having any combination of any of the foregoing. Examples of suitable ligands include mannose, immunoglobulins, CpG, integrin motifs and any combination thereof. The formulation comprising the binding-deficient extoxin may include one or more different types of binding deficient exotoxins, further described herein in detail. Additionally, the formulation comprising the binding-deficient exotoxin may further include at least one exotoxin molecule which is not a binding-deficient exotoxin.

Addition of a bacterial ADP-ribosylating exotoxin (bARE) to a formulation (e.g., immunogen or vaccine) or a system (e.g., patch or kit) for immunization enhances the immune response in a subject to one or more components of the formulation. In the case of LT and CT, in vivo binding of the B subunit of a bARE to cell-surface receptors on a subject's cells, however, mediates the toxicity of the A subunit and limits the use of native bARE as adjuvants. Mutation of the B subunit or in vitro coupling of the B subunit to cognate receptors or chemical ligands, such as ganglioside GM1 or other gangliosides, α₂-macroglobulin receptor, low density lipoprotein receptor-related protein (LRP), or a B subunit-binding portion thereof, inhibits binding to certain cell-surface receptors in vivo, thereby eliminates toxicity but retains desired adjuvant activity. The use of these exotoxins provides a safe and potent new strategy for development of effective formulations for immunization and vaccination and inducing an antigen specific immune response.

Embodiments of the invention include products containing GM-1 binding deficient exotoxins, their production, and their use in immunization, inducing an antigen specific immune response and vaccination. The cognate receptor may be any portion of the cell-surface receptor that binds to the exotoxin's B subunit. The toxin-binding ligand maybe any chemical structure blocking subsequent binding of the B subunit to gangliosides in vivo. Also, the invention includes the use of exotoxins with any mutation of the B subunit leading to loss or attenuation of binding to endogenous GM1 or other gangliosides. The invention embodies the use of these non-toxic exotoxin formulations in diverse delivery strategies including intradermal, intramuscular, subcutaneous and topical administration and are useful in vaccine formulations and methods of inducing immune responses to a wide variety of antigens. Topical administration as used herein does not include nasal or intranasal administration. Exotoxin formulations include usage in creams, gels, hydrogels, emulsions, liposomes, spray dried formulations, sprays, or injection fluids. Dry formulations may be provided in various forms: for example, fine or granulated powders, uniform films, pellets, and tablets. The formulation may be dissolved and then dried in a container or on a flat surface (e.g., skin), or it may simply be dusted on the flat surface. It may be air dried, dried with elevated temperature, freeze or spray dried, coated or sprayed on a solid substrate and then dried, dusted on a solid substrate, quickly frozen and then slowly dried under vacuum, or combinations thereof. If different molecules are active ingredients of the formulation, they may be mixed in solution and then dried, or mixed in dry form only.

Exotoxin formulations are included in adhesive patches and other devices or methods for topical delivery (WO 99/43350, WO 00/61184 and WO 02/74325). For parenteral delivery, exotoxin formulations are included in pressurized container devices or syringes and microneedles. Further aspects of the invention will be apparent to a person skilled in the art from the following detailed description and claims, and generalizations thereto.

Adjuvants are substances that stimulate antigen-specific immune responses. An antigen is defined as a substance that induces a specific immune response when presented to immune cells of an organism. Adjuvants may be chosen to induce specific components of the immune system (Edelman, 2000), such as specific antibody or antibody subset responses (e.g., IgG1, IgG2, IgM, IgD, IgA, IgE) or T cell responses (e.g., CTL, Th1, Th2).

Adjuvants are added to antigen formulations to enhance the immune response to the antigen. The formulation can either be a combination of an antigen and an adjuvant or separate formulations of the antigen and the adjuvant. For example, a first formulation can comprise at least one GM-1 binding deficient exotoxin while a second formulation can comprise at least one antigen. Formulations can be applied via intramuscular, intradermal, subcutaneous, or topical routes. When antigens come in contact with the immune system, an immune response can be induced directly or through an antigen presenting cell (e.g., macrophages, Langerhans cells, other dendritic cells, B cells) that presents processed antigens to T cells. Langerhans cells and dermal dendritic cells are the most potent antigen presenting cells in the skin (Udey, 1997). Adjuvants are assumed to enhance immune responses by, for example, targeting the antigen to antigen presenting cells (APC), increasing antigen uptake and processing by APC, enhancing presentation to T cells, or combinations thereof (Udey, 1997; Glenn et al., 2000).

Bacterial exotoxins from the family of ADP-ribosylating toxins (bAREs) are potent stimulators of humoral- and cell-mediated immune responses to themselves and to coadministered antigens (Snider, 1995). Examples of bAREs are cholera toxin (CT), E. coli heat-labile enterotoxin (LT), diphtheria toxin (DT), pertussis toxin (PT), and P. aeruginosa exotoxin A (ETA). Many bAREs are composed of subunits containing a cell membrane binding B subunit and an A subunit exerting ADP-ribosylation activity. The B subunit of CT and LT binds to ganglioside GM1 located on the cell surface of mammalian cells. Binding to cell surface GM1 opens an aqueous membrane pore allowing the A subunit to gain access to the cytoplasm enabling it to execute its ADP-ribosylating activity. ADP-ribosylation of a G protein (G_{sα}) involved in activation of the adenylate cyclase system results in persistent synthesis of cAMP causing toxicity (Sears & Kaper, 1996). Thus, binding to cell surface GM1 is an essential first step in the mechanism underlying toxicity of exotoxins.

GM1 (Gal-β1,3-GalNAc-β1,4-(NeuAc-α2,3)-Gal-β1,4-Glc-β1,1-ceramide) is a ubiquitous cell membrane ganglioside. It is the predominant receptor on cell surfaces for binding the B pentamer with high affinity. The oligosaccharide part of GM1 is responsible for exotoxin binding. The three dimensional crystal structure of LT and the exotoxin-GM1 complex are known (Sixma et al., 1991; Merritt et al., 1998). The two terminal sugars, galactose and sialic acid, make the most contributions to the binding of GM1 to the exotoxins. LRP, the α₂-macroglobulin receptor-low density lipoprotein receptor-related protein, is a receptor for P. aeruginosa exotoxin A (ETA). As proposed in this invention, a new approach would be to block or attenuate binding to GM1 in vivo to prevent toxicity while retaining adjuvant activity. This can be achieved by mutation of the B subunit or binding of exotoxins in vitro with ligands and/or cognate receptors or both to block subsequent interaction of the B subunit with GM1 in vivo.

Activation of dendritic cells is a key factor in the adjuvant properties of exotoxins (Glenn et al., 2000). Dendritic cells express on their cell surface specific molecules or receptors which distinguish them from surrounding cells in the tissue. Therefore, further modification of the B subunit with specific ligands for dendritic cell receptors may increase targeting of the exotoxin to the dendritic cells. Langerhans cells in the skin express mannose receptors, Fc receptors, and lectins, which play a role in antigen binding and uptake by endocytosis (de la Salle et al., 1997; Condaminet et al., 1998; Dong et al., 1999; Valladeau et al., 2000). Thus, further modification of the B subunit by mannosylation, linkage to immunoglobulin fragments, or linkage to lectin-binding integrins will increase specific delivery to the dendritic cells and decrease nonspecific targeting of irrelevant bystander cells, contributing to less toxicity while retaining beneficial immune activation (U.S. Pat. Nos. 5,807,988 and 6,046,158).

Coupling of bacterial exotoxins to GM1 in vitro is achieved by incubation of the exotoxin with GM1 in saline for 60 min. at room temperature. For other types of substrates, a change in incubation time, pH, ionic strength, or temperature may be required for optimal binding to the exotoxin. Coupling of some substrates may require methods which include chemical or enzymatic coupling agents to facilitate conjugation, such as tyrosine oxidation with the Ni(II) complex of the tripeptide GGH and a peroxide oxidant, crosslinking of reactive Lys and Gln residues with specific peptidyl linkers, dextran polyaldehyde mediated protein cross linking, or N-succinimidyl 3-(2-pyridyldithio) propionate (SPDP) mediated conjugation linked via disulfide bonds.

In vitro complete or partial inactivation of the binding site within the B subunit by mutation or binding to their receptor counterparts (e.g., GM1, GM1 derivatives, partial GM1 molecules, GM2, GM3, GD2, GD3, GD1b and other gangliosides) will markedly decrease in vivo binding and thereby attenuate toxicity. The binding molecule can be any one of a ganglioside, a B subunit-binding portion of a ganglioside, a low density lipoprotein receptor-related protein (LRP), a B subunit-binding portion of LRP, an alpha macroglobulin receptor, and a B subunit-binding portion of an alpha macroglobulin receptor. Inactivation of the binding site may also be achieved by high affinity chemical substrates such as modification of free amino groups by poly(ethylene)glycol or alkylation of free carboxylic groups by acetic anhydride. A trace amount of holotoxin may be left in the formulation since this may add some benefit to adjuvant activity without toxicity (Tamura et al., 1994). In addition, small nontoxic amounts of contact sensitizers or LPS derivatives (e.g., lipid A, CpG) may be added to enhance adjuvant activity. Exotoxins containing a mutation in the GM1 binding B subunit are described (Nashar et al, 1996) resulting in complete loss of binding to GM1. E.g., a G33D mutant contains a single amino acid substitution, i.e., aspartate for glycine at residue 33. The larger size of the side chain at residue 33 did not play a role, because substitution with an even larger arginine retained GM1-binding (Merritt et al, 1997). The critical nature of the side chain of residue 33 is apparently due to a limited range of subtle rearrangements available to both the toxin and the saccharide to accommodate receptor binding (Merritt et al, 1997). One B subunit contains a sequence of 103 amino acid residues. Other residues than G33 are critical in GM1 binding, such as Tyr-12, and mutation of these positions create non-toxic, non-GM1 binding mutants. This invention embodies any B subunit mutation at any position leading to loss of binding to GM1.

Several studies with a non-GM1 binding mutant failed to show potent immunomodulatory effects supporting the assumption of GM 1-binding to be essential for exotoxin adjuvanticity (Nashar et al, 1996; Guidry et al., 1997). Unexpectedly, we found that G33D mutants and GM1-LT complexes retain adjuvanticity. The adjuvanticity was observed by transcutaneous immunization (TCI), immunostimulant patch strategies accompanying intradermal, subcutaneous and intramuscular immunizations, and with parenteral immunization with antigen and adjuvant. It was unexpected to find that the adjuvanticity is independent of in vivo binding to GM1. We envision that reduced binding to the ubiquitously expressed GM1 may result in reduced cellular uptake of exotoxins to levels which are non-toxic but still sufficient to induce immunostimulatory effects. However, alternative cellular binding and uptake mechanisms may also exist, targeting exotoxins to more discrete cell populations with immunostimulatory capacity, thereby evading injury of bystander cells.

The effect of Escherichia coli infection of mammals is dependent on the particular strain of organism. Many beneficial E. coli are present in the intestines. Since the initial association with diarrheal illness, five categories of diarrheagenic E. coli have been identified: enterotoxigenic Escherichia coli (ETEC), enteropathogenic Escherichia coli (EPEC), enterohemorrhagic Escherichia coli (EHEC), enteroaggregative Escherichia coli (EAggEC), and enteroinvasive Escherichia coli (EIEC).

They are grouped according to characteristic virulence properties, such as elaboration of toxins and colonization factors and/or by specific types of interactions with intestinal epithelial cells. ETEC are the most common of the diarrheagenic E. coli and pose the greatest risk to travelers. Strains which have been cultured from humans include 137A (CS6, LT, STa), H10407 (CFA/1, LT, STa) and E24377A (CS3, CS1, LT, STa).

They may be used singly or in combination as whole-cell sources of antigen providing a variety of different toxins and colonization factors. There is a need for vaccines which are specific against enterotoxigenic E. coli that give rise to antibodies that cross-react with and cross-protect against the more common colonization and virulence factors. The CS4-CFA/I family of fimbrial proteins are found on some of the more prevalent enterotoxigenic E. coli strains: there are six members of this family of ETEC antigens, CFA/I, CS1, CS2, CS4, CS17, and PCIF 0166.

Colonization factor antigens (CFA) of ETEC are important in the initial step of colonization and adherence of the bacterium to intestinal epithelia. In epidemiological studies of adults and children with diarrhea, CFA/I is found in a large percentage of morbidity attributed to ETEC. The CFA/1 is present on the surfaces of bacteria in the form of pili (fimbriae), which are rigid, 7 nm diameter protein fibers composed of repeating pilin subunits. The CFA/I antigens promote mannose-resistant attachment to human brush borders with an apparent sialic acid sensitivity. Hence, it has been postulated that a vaccine that establishes immunity against these proteins may prevent attachment to host tissues and subsequent disease.

Other antigens including CS3, CS5, and CS6. CFA/I, CS3 and CS6 may occur alone, but with rare exception CS1 is only found with CS3, CS2 with CS3, CS4 with CS6 and CS5 with CS6. Serological studies show these antigens occur in strains accounting for up to about 75% or as little as about 25% of ETEC cases, depending on the location of the study.

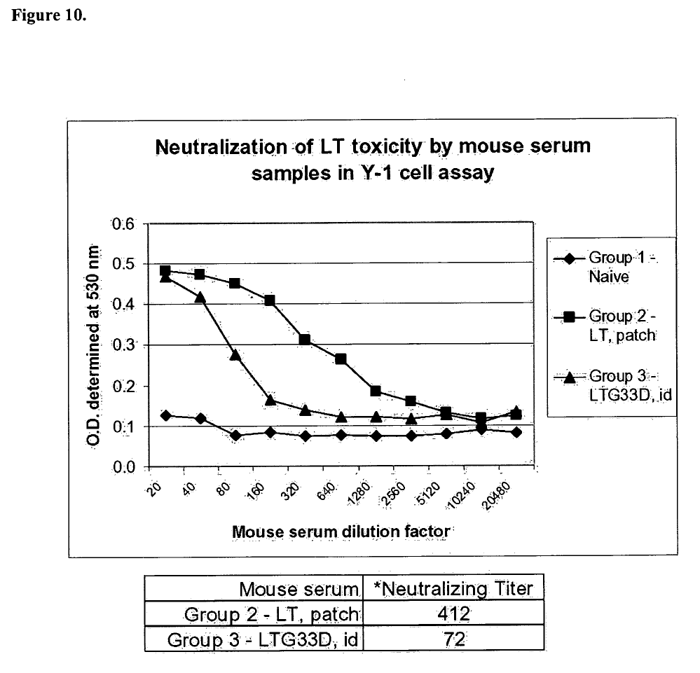

Another approach to development of a vaccine against ETEC is to target the enterotoxins responsible for clinical disease. Two enterotoxins are produced by ETEC and are designated as heat stable toxin (ST) or heat labile toxin (LT). One or both toxins may be produced by different strains of ETEC. Of the ETEC strains that infect humans, 75% produce ST and over 50% produce LT. Because these toxins are reactogenic in humans, a vaccine to these toxins has not been possible. Therefore, within the context of this disclosure, we have demonstrated that it is feasible to use the non-reactogenic LT Gly33Asp as a vaccine for producing antibodies that will neutralize the toxic effects of wild type LT. In this study, mice were immunized by intradermal injection with 0.5 μg of LTGly33Asp and a separate group was topically immunized with 25 μg of wild type LT. Both groups were immunized on three times (day 1, 15 and 29) and serum was collected two weeks after the third immunization. LT neutralizing antibody titers were determined by inhibition of LT cytotoxicity in the Y1 cell assay. As seen in FIG. 10, mice immunized with the mutant LT had high titer antibodies that effectively neutralized the cytotoxic activity of wild type LT. These results support the concept that the non-reactogenic mutant LT can be administered without side affects and elicit the generation of high titer antibodies which are toxin neutralizing.

Formulations

Formulation in liquid or solid form may be applied with one or more adjuvants and/or antigens both at the same or separate sites or simultaneously or in frequent, repeated applications. When the antigen and exotoxin are formulated in separate formulations, they can be administered via distinct routes at the same or separate sites.

This invention embodies usage of ganglioside-binding deficient bARE's as adjuvant in parenteral and topical vaccine formulations. Topical applications include patch delivery systems. A “patch” refers to a product which includes a solid substrate (e.g., occlusive or nonocclusive surgical dressing) as well as at least one active ingredient. Liquid may be incorporated in a patch (i.e., a wet patch). One or more active components of the formulation may be applied on the substrate, incorporated in the substrate or adhesive of the patch, or combinations thereof. A liquid formulation may be held in a reservoir or may be mixed with the contents of a reservoir. A dry patch may or may not use a liquid reservoir to solubilize the formulation. Compartments or chambers of the patch may be used to separate active ingredients so that only one of the antigens or adjuvants is kept in dry form prior to administration; separating liquid and solid in this manner allows control over the time and rate of the dissolving of at least one dry, active ingredient. Similarly, the adjuvant and antigen may be applied as separate patches.

The patch may include a controlled-release reservoir or a rate-controlling matrix or membrane may be used which allows stepped release of adjuvant and/or antigen. It may contain a single reservoir with adjuvant and/or antigen, or multiple reservoirs to separate individual antigens and adjuvants. The patch may include additional antigens such that application of the patch induces an immune response to multiple antigens. In such a case, antigens may or may not be derived from the same source, but they will have different chemical structures so as to induce an immune response specific for different antigens. Multiple patches may be applied simultaneously; a single patch may contain multiple reservoirs. For effective treatment, multiple patches may be applied at intervals or constantly over a period of time; they may be applied at different times, for overlapping periods, or simultaneously. At least one adjuvant and/or adjuvant may be maintained in dry form prior to administration. Subsequent release of liquid from a reservoir or entry of liquid into a reservoir containing the dry ingredient of the formulation will at least partially dissolve that ingredient.

Solids (e.g., particles of nanometer or micrometer dimensions) may also be incorporated in the formulation. Solid forms (e.g., nanoparticles or microparticles) may aid in dispersion or solubilization of active ingredients; assist in carrying the formulation through superficial layers of the skin; provide a point of attachment for adjuvant, antigen, or both to a substrate that can be opsonized by antigen presenting cells, or combinations thereof. Prolonged release of the formulation from a porous solid formed as a sheet, rod, or bead acts as a depot.

The formulation may be manufactured under conditions acceptable to appropriate regulatory agencies (e.g., Food and Drug Administration) for biologicals and vaccines. Optionally, components like binders, buffers, colorings, dessicants, diluents, humectants, preservatives, stabilizers, other excipients, adhesives, plasticizers, tackifiers, thickeners, patch materials, or combinations thereof may be included in the formulation even though they are immunologically inactive. They may, however, have other desirable properties or characteristics which improve the effectiveness of the formulation.

A single or unit dose of formulation suitable for administration is provided. The amount of adjuvant or antigen in the unit dose may be anywhere in a broad range from about 0.001 μg to about 10 mg. This range may be from about 0.1 μg to about 1 mg; a narrower range is from about 5 μg to about 500 μg. Other suitable ranges are between about 1 μg and about 10 μg, between about 10 μg and about 50 μg, between about 50 μg and about 200 μg, and between about 1 mg and about 5 mg. A preferred dose for a toxin is about 50 μg or 100 μg or less (e.g., from about 1 μg to about 50 μg or 100 μg). The ratio between antigen and adjuvant may be about 1:1 (e.g., an ADP-ribosylating exotoxin when it is both antigen and adjuvant) but higher ratios may be suitable for poor antigens (e.g., about 1:10 or less), or lower ratios of antigen to adjuvant may also be used (e.g., about 10:1 or more). The dose of antigen and/or adjuvant to be administered is easily determined by one of ordinary skill in the art using well known methods and techniques.

A formulation comprising adjuvant and antigen or polynucleotide may be applied to skin of a human or animal subject, antigen is presented to immune cells, and an antigen-specific immune response is induced. This may occur before, during, or after infection by pathogen. Only antigen or polynucleotide encoding antigen may be required, but no additional adjuvant, if the immunogenicity of the formulation is sufficient to not require adjuvant activity. The formulation may include an additional antigen such that application of the formulation induces an immune response against multiple antigens (i.e., multivalent). In such a case, antigens may or may not be derived from the same source, but the antigens will have different chemical structures so as to induce immune responses specific for the different antigens. Antigen-specific lymphocytes may participate in the immune response and, in the case of participation by B lymphocytes, antigen-specific antibodies may be part of the immune response. The formulations described above may include binders, buffers, colorings, dessicants, diluents, humectants, preservatives, stabilizers, other excipients, adhesives, plasticizers, tackifiers, thickeners, and patch materials known in the art.

In addition to antigen and adjuvant, the formulation may comprise a vehicle. For example, the formulation may comprise an AQUAPHOR, Freund, Ribi, or Syntex emulsion; water-in-oil emulsions (e.g., aqueous creams, ISA-720), oil-in-water emulsions (e.g., oily creams, ISA-51, MF59), microemulsions, anhydrous lipids and oil-in-water emulsions, other types of emulsions; gels, fats, waxes, oil, silicones, and humectants (e.g., glycerol). Many other vehicles known to those of ordinary skill in the art may be used and are envisioned in the practice of the invention.

Antigen may be solubilized in a buffer or water or organic solvents such as alcohol or DMSO, or incorporated in gels, emulsions, lipid micelles or vesicles, and creams. Suitable buffers include, but are not limited to, phosphate buffered saline Ca⁺⁺/Mg⁺⁺ free, phosphate buffered saline, normal saline (150 mM NaCl in water), and Hepes or Tris buffer. Antigen not soluble in neutral buffer can be solubilized in 10 mM acetic acid and then diluted to the desired volume with a neutral buffer such as PBS. In the case of antigen soluble only at acid pH, acetate-PBS at acid pH may be used as a diluent after solubilization in dilute acetic acid. Dimethyl sulfoxide and glycerol may be suitable nonaqueous buffers for use in the invention.

A hydrophobic antigen can be solubilized in a detergent or surfactant, for example a polypeptide containing a membrane-spanning domain. Furthermore, for formulations containing liposomes, an antigen in a detergent solution (e.g., cell membrane extract) may be mixed with lipids, and liposomes then may be formed by removal of the detergent by dilution, dialysis, or column chromatography. Certain antigens (e.g., membrane proteins) need not be soluble per se, but can be inserted directly into a lipid membrane (e.g., virosome), in a suspension of virion alone, or suspensions of microspheres or heat-inactivated bacteria which may be taken up by activate antigen presenting cells (e.g., opsonization). Antigens may also be mixed with a penetration enhancer as described in WO 99/43350.

Processes for manufacturing a pharmaceutical formulation are well known. The components of the formulation may be combined with a pharmaceutically-acceptable carrier or vehicle, as well as any combination of optional additives (e.g., at least one binder, buffer, coloring, dessicant, diluent, humectant, preservative, stabilizer, other excipient, or combinations thereof). See, generally, Ullmann's Encyclopedia of Industrial Chemistry, 6^{th} Ed. (electronic edition, 1998); Remington's Pharmaceutical Sciences, 22^{nd} (Gennaro, 1990, Mack Publishing); Pharmaceutical Dosage Forms, 2^{nd} Ed. (various editors, 1989-1998, Marcel Dekker); and Pharmaceutical Dosage Forms and Drug Delivery Systems (Ansel et al., 1994, Williams & Wilkins).

Good manufacturing practices are known in the pharmaceutical industry and regulated by government agencies (e.g., Food and Drug Administration). A liquid formulation may be prepared by dissolving an intended component of the formulation in a sufficient amount of an appropriate solvent. Generally, dispersions are prepared by incorporating the various components of the formulation into a vehicle which contains the dispersion medium. For production of a solid form from a liquid formulation, solvent may be evaporated at room temperature or in an oven. Blowing a stream of nitrogen or air over the surface accelerates drying; alternatively, vacuum drying or freeze drying can be used. Solid dosage forms (e.g., powders, granules, pellets, tablets), liquid dosage forms (e.g., liquid in ampules, capsules, vials), and patches can be made from at least one active ingredient or component of the formulation.

Suitable procedures for making the various dosage forms and production of patches are known. The formulation may also be produced by encapsulating solid or liquid forms of at least one active ingredient or component, or keeping them separate in compartments or chambers. The patch may include a compartment containing a vehicle (e.g., saline solution) which is disrupted by pressure and subsequently solubilizes the dry formulation of the patch. The size of each dose and the interval of dosing to the subject may be used to determine a suitable size and shape of the container, compartment, or chamber.

The relative amounts of active ingredients within a dose and the dosing schedule may be adjusted appropriately for efficacious administration to a subject (e.g., animal or human). This adjustment may depend on the subject's particular disease or condition, and whether therapy or prophylaxis is intended. To simplify administration of the formulation to the subject, each unit dose would contain the active ingredients in predetermined amounts for a single round of immunization.

Methods for Examples

Immunization Procedures

For transcutaneous immunization, mice (e.g., BALB/c, C57BL/6, DBA) aged 6 to 8 weeks are shaved without any signs of trauma to the skin (e.g., wounds, irritation). The shaven area may be the back, abdomen, neck, or leg. Prior to shaving, the mice are ear tagged for identification and bled to obtain pre-immunization sera. Two days after shaving, the shaven skin is hydrated by rubbing with a saline-wetted gauze. Five minutes after hydration, the immunization solution containing the formulation of adjuvant and antigen is pipetted onto the shaven skin. After contact for one hour, the shaven area is washed under a stream of tap water. As an alternative to pipetting liquid onto the skin, delivery of antigen-adjuvant formulations may be achieved by an overnight skin patch containing the formulation.

For immunization of humans, a gauze pad containing the antigen-adjuvant formulation, under an adhesive patch (WO00/61184, WO02/74325), may be applied to hydrated skin on the upper arm for six hours. In humans shaving is not required.

In addition, skin may be pretreated by stripping with adhesive tape or abrasion with pumice, emery board, sandpaper, or other techniques to enhance delivery (WO99/43350). For tape stripping, for example, adhesive tape (3M mailing tape, Staples #8958) is attached firmly to the skin and ripped off quickly. This procedure is repeated five to ten times in different directions with a new piece of tape each time. For abrading the skin, for example, fine-grade sandpaper (GE Medical Systems, #E9001CK) or a pumice pad (Electrode Prep Pads, PDI #B59800) is applied to the skin under slight pressure and rubbed five to ten times in the same direction.

GM-1 binding deficient exotoxins may also be used in intramuscular, intradermal, and subcutaneous administration. Small volumes of adjuvant/antigen formulations (e.g., 25 μl to 100 μl in mice) are injected/inserted in the dermis, in the muscle, or beneath the dermis.

Antibody Responses

Antibodies specific for the adjuvant or antigen is determined using ELISA. Antigens or adjuvants are dissolved in PBS at a concentration of 2 μg/ml; 50 μl of solution per well is applied to IMMULON-2 polystyrene plates and incubated at room temperature overnight. Then the plates are blocked with 0.5% casein/1% Tween 20 in PBS (i.e., blocking buffer) for one hour. Sera or other body fluids are diluted with 0.5% casein/0.025% Tween 20 in PBS, transferred to the plates, and incubated for 2 hr or overnight. Then plates are washed with PBS containing 0.05% Tween 20 and incubated with goat anti-mouse IgG horseradish peroxidase (HRP)-linked secondary antibody for one hour at room temperature. Plates are washed again, incubated with HRP substrates, and color development is analyzed at 405 nm by spectrophotometry. Similar procedures are applied to determine specific subclasses of antibodies (e.g., IgG1, IgG2a, IgG2b, IgM, IgA) by using appropriate subclass-specific reagents.

CTL Proliferation

Spleen and/or lymph nodes are isolated from mice and single cell suspensions are generated by forcing tissue through a nylon mesh. Cells are suspended in tissue culture medium (48.5% v/v RPMI-1640, 48.5% v/v EHAA Click's medium, 2 mM Glutamine, 1% v/v Pen/Strep solution, 0.5% v/v autologous normal mouse serum, 0.1% v/v β-mercaptoethanol, and 0.5% v/v 1M HEPES). Cells are cultured with antigen for 4 days in a CO₂ incubator at 37° C. and then incubated for 16 hr to 18 hr with 1 μCi of [³H]-thymidine in 50 μl. Proliferation is measured by incorporation of radioactive thymidine.

ELISPOT

Nitrocellulose-backed microtiter plates are coated overnight at 4° C. with cytokine-specific primary antibody in PBS. Then plates are blocked with PBS containing 0.5% w/v BSA at 37° C. for 30 min. Plates are washed with RPMI-1640 and 10% v/v FBS medium. Cell suspensions isolated from mice spleen and/or lymph nodes are added to the plates in serial dilutions and antigen is added to the cells for 6 to 24 hr at 37° C. in a CO₂ incubator. Then plates are washed with PBS containing 0.025% v/v Tween 20 followed by distilled water to lyse the cells. Biotin-labeled anti-cytokine antibody is added to the plates followed by washing and subsequent incubation with alkaline phosphatase-labeled avidin D. Plates are washed again and spots are visualized with substrate (BCIP/NBT solution). The number of spots are counted which reflects the number of specific cytokine-secreting cells.

Evaluation of Toxicity in Vivo Using a Model of Fluid Accumulation

Naive and/or immunized mice are challenged orally with exotoxin adjuvants suspended in 500 μl of 10% w/v sodium bicarbonate (NaHCO₃) solution. Control animals received 500 μl of 10% NaHCO₃ alone. To prevent coprophagy, the mice are transferred to cages with wire mesh flooring. Mice are fasted for 12 hr before challenge and during challenge. Six hours after the challenge, the animals are weighed and sacrificed. The small intestines are then dissected (pyloric valve to ileal-cecal junction), tied off to prevent fluid loss, and weighed. Toxicity was determined by measuring intestinal fluid accumulation relevant to body weight (Yu et al., 2002).

Ganglioside Binding

Gangliosides (e.g., GM1, GM2, other) are obtained from animal (e.g., bovine) or human sources (Svennerholm in Methods in Carbohydrate Chemistry, Vol. VI, 464). Binding of exotoxins to gangliosides is achieved in vitro by mixing both together in PBS followed by 90 min incubation at 37° C. before use as an adjuvant. The amount of ganglioside bound may be varied by using a range of exotoxin to ganglioside molar ratios (i.e., 1:0.5 to 1:500). Exotoxin-binding to gangliosides can be measured by ELISA:ganglioside-coated ELISA plates are blocked with 2% w/v BSA and 0.05% v/v Tween 20 in PBS for one hour at room temperature. Plates are washed with washing buffer (0.05% Tween in PBS) and test samples are added to the plates for incubation overnight. Then plates are washed again and incubated with anti-exotoxin antibody. Plates are washed again followed by incubation with goat anti-mouse IgG-HRP. After washing, substrate is added and the amount of exotoxin bound to the plates is quantified.

In Vitro Analyses of Exotoxin Biological Activity

Y-1 adrenal cells are grown in Ham F12 medium supplemented with 2 mM glutamine, 100 U/ml penicillin, 100 μg/ml streptomycin, and 10% v/v FBS (37 ° C./5% CO₂). For the experimental assay, cells are trypsinized, replated in 96 well plates at 2×10⁴ cells per well, and cultured for another 3-5 days. Then, cells are incubated with medium containing various concentrations of exotoxin for 6-8 h, and rounding of the cells is determined under an inversion microscope.

### EXAMPLES

The following examples are merely illustrative of embodiments described in the invention herein, and are not intended to restrict or otherwise limit its practice.

### Example 1

### The Enterotoxicity of AB5 Toxin is Attenuated by Blocking in Vivo Binding to GM1 Ganglioside Receptors with a High Affinity Receptor Antagonist

To evaluate the toxicity of the modified adjuvants, naive Balb/c mice were orally challenged with a sublethal dose of the exotoxin and swelling of the small intestine was determined as a measure of toxicity (Yu et al., 2002). The gut to carcass ratio was determined by removing and weighing the intestine and carcass. In this study, adult BALB/c mice were fasted overnight. Immediately before challenge, mice were fed sodium bicarbonate solution to neutralize stomach acid. Mice were then challenged by oral administration of 25 μg LT or with LT/GM-1 complex. After 6 hours, animals were sacrificed and a ligature was used to tie both ends of the small intestine before removal. The intestines and carcass were weighed and the gut to carcass ratio (G:C) calculated. The results in Table 1 show the mean G:C of groups of 10 mice. The G:C for non-treated control mice was 0.054. Mice fed 25 μg LT had a 2.4-fold increase in G:C (0.13) compared to the buffer control group. Mice fed 25 μg LT complexed with an excess of GM-1 ganglioside (37.5 μg or 6.25 μg) had a G:C equal to the non-challenged control group (G:C=0.052 and 0.057, respectively). Reducing the GM-1 ganglioside to 1.25 μg had a G:C modest accumulation of water (1.6-fold compared to the buffer treated group) in the intestine (G:C=0.085), while the group receiving 25 μg LT complexed with very low doses of GM-1 ganglioside (0.25 μg) had a G:C ratio (0.123) equivalent to the group treated with 25 μg LT without GM-1. These results demonstrate that LT enterotoxicity may be completely inhibited by blocking all five receptor-binding sites on the B-subunit when an excess molar ratio of a high affinity receptor antagonist was used. Partial attenuation was achieved by blocking as few as 3-receptor binding domains, while blocking 1 binding site had no significant effect on enteroxicity. Accordingly, a similar study conducted previously demonstrated no intestinal swelling using the non-GM1 binding mutant LTGly33Asp (Guidry et al, 1997).

TABLE 1
Oral challenge of mice with LT or LT-GM1

| | | | LT/GM1 | Intestinal Swelling |
|---|---|---|---|---|
| | Oral | Receptor | (molar | Weight |
| Group | Challenge | Antagonist | ratio) | (Gut/Carcass) |
| 1 | buffer | — | — | 0.054 ± 0.005 |
| 2 | LT (25 μg) | — | — | 0.131 ± 0.022 |
| 3 | LT (25 μg) | GM1 (37.5 μg) | 1:83 | 0.052 ± 0.004 |
| 4 | LT (25 μg) | GM1 (6.25 μg) | 1:14 | 0.057 ± 0.007 |
| 5 | LT (25 μg) | GM1 (1.25 μg) | 1:2.8 | 0.085 ± 0.014 |
| 6 | LT (25 μg) | GM1 (0.25 μg) | 1:0.6 | 0.123 ± 0.027 |

Balb/c mice were given 500 μl buffer (10% NaHCO₃ in water) alone or mixed with LT and various amounts of GM1. Six hours after challenge the weight of the small intestine was measured relative to the body weight. Data represent mean ± SD of 10 mice per group.

### Example 2

### Attenuation of AB5-Induced Cutaneous Inflammation by Blocking in Vivo Binding to GM1 Ganglioside with a High Affinity Receptor Antagonist

LT is highly reactogenic when injected neat into the dermis or subcutaneous tissues. Injected LT elicits erythema and swelling at the site of injection, which in time becomes raised and indurated and may persist for longer than one week. Therefore, another way to evaluate toxicity is by measuring of skin swelling (formation of skin nodules) in response to intradermal injection with LT. For example, intradermal injection of 0.5 μg LT caused skin nodules in all mice with an average diameter of 1.24 cm (Table 2, group 1). Injection of 0.5 μg LT together with 0.075 μg soluble GM1 ganglioside elicited an inflammatory response in 4 of 7 mice (Table 2, group 2), while injection of 0.5 μg LT with 0.75 μg of soluble GM1 did not cause skin nodules in six out of the seven injected mice (Table 2, group 3). Soluble GM1 ganglioside was not inflammatory (group 4).

Full thickness biopsies were taken for histological examination following intradermal injection with LT and LT/GM-1. Consistent with the gross observations, biopsies obtained from mice 1-2 days after injection were edematous with diffuse polymorphonuclear leukocytes cells throughout the epidermis, dermis and subcutaneous tissues. Within several days, the injection sites were infiltrated by mononuclear cells throughout the epidermis, dermis and subcutaneous tissue layers. In contrast, histologic examination of LT/GM-1 injected skin exhibited normal skin architecture and an absence of inflammatory cells. Based upon gross and histological examination, LT/GM-1 did not elicit significant inflammatory responses when injected ID. In contrast, LT injection elicited an inflammatory response that persisted for days. In summary, LT enterotoxicity and skin reactogenicity is attenuated when the GM1 binding domain of the B-subunit is unable to interact with the high affinity receptor in vivo. In Examples 1 and 2, we used soluble GM1 ganglioside as a high affinity receptor antagonist to occupy the B-subunit receptor binding pockets. Using this strategy, we demonstrated that the inflammatory properties of LT were ameliorated or completely prevented by pre-adsorption of LT with GM1 ganglioside before administration. The in vivo toxicity of LT, and likely other AB5 extoxins, is attenuated by blocking in vivo binding to high affinity GM1 ganglioside receptors. Formulating extoxins with a receptor antagonist is an effective method to attenuate toxicity.

TABLE 2
Skin nodule formation after intradermal injection of LT or LT-GM1

| | Intra- | Assay values 1-7 | | | | | | | |
|---|---|---|---|---|---|---|---|---|---|
| | dermal | Diameter skin nodules (cm) | | | | | | | |
| | injection | Individual Mice | | | | | | | Average |
| Groups | (μg) | 1 | 2 | 3 | 4 | 5 | 6 | 7 | Mean ± SD |
| 1 | LT (0.5 | 1.1 | 1.3 | 1.3 | 1.0 | 1.4 | 1.25 | 1.3 | 1.24 ± 0.13 |
| | μg) | | | | | | | | |
| 2 | LT (0.5) + | 0 | 0 | 0.7 | 0 | 0.7 | 0.65 | 0.75 | 0.4 ± 0.35 |
| | GM1 | | | | | | | | |
| | (0.075) | | | | | | | | |
| 3 | LT (0.5) + | 0 | 0 | 0 | 0 | 0 | 0 | 0.95 | 0.14 ± 0.33 |
| | GM1 | | | | | | | | |
| | (0.75) | | | | | | | | |
| 4 | GM1 | 0 | 0 | 0 | 0 | 0 | 0 | 0 | 0 |
| | (0.75) | | | | | | | | |

C57B1/6 mice received an intradermal injection of 25 μl PBS containing LT and/or GM1. The next day, swelling of the skin was measured.

### Example 3

Attenuation of AB5 Toxin Induced Inflammation by Mutations that Disrupt GM1 Ganglioside Binding

Another approach to demonstrate the association between in vivo receptor binding and toxicity is to use a mutant variant of LT that is unable to bind to GM1 ganglioside receptors. Nonspecific and site directed mutagenesis has been used to generate a mutant form of LT that does not bind to the GM1 ganglioside receptor. The mutant LT has a single residue substitution in position 33 of the B subunit, where Gly as been replaced with Asp. The mutant, LTGly33Asp (LTG33D), does not bind the GM1 ganglioside receptors (Tsuji et al., 1985 and Guidry et al., 1997). As described in Example 2, intradermal injection of 0.5 μg of wild type LT causes an inflammatory response that is manifested as raised, indurated nodules (1.04 cm diameter) at the site of injection. Injection of the same amount of LT-Gly33Asp or vehicle (phosphate suffered saline, PBS) did not cause a nodule to develop at the injection. Histological evaluation of the LT-Gly33Asp-injected site showed no significant edema or inflammation, such as erythema, unlike wild type LT (Table 3). As demonstrated by two different approaches, LT toxicity is mediated through GM1 ganglioside binding. Enterotoxicity and skin reactogenicity is attenuated by perturbing receptor binding in vivo.

TABLE 3
Skin nodule formation after intradermal injection of LT or LT-Gly33Asp

| Group | Intradermal injection | Skin Nodules (cm) |
|---|---|---|
| 1 | PBS | 0 |
| 2 | LT (0.5 μg) | 1.04 ± 0.15 |
| 3 | LT-Gly33Asp (0.5 μg) | 0.06 ± 0.12 |

C57B1/6 mice received an intradermal injection of 25 μl PBS alone, or mixed with LT/LT-Gly33Asp. The next day swelling of the skin was measured. Data shown are means ± SD of 10 mice per group.

### Example 4

### Lack of Reactogenicity of LTGly33Asp and LT/GM1 when Administered by Subcutaneous and Intramuscular Injection

An additional study was conducted to investigate the potential for use of LTGly33Asp and LT/GM1 complex by other parenteral routes including, intramuscular (IM) and subcutaneous (SC). In this study, mice were injected with 0.5 μg wild type LT under the skin or into the thigh muscle. As observed with ID injection, the wild type LT elicited the formation of a large nodule with SC injection and swelling of the thigh muscle with IM injection. In contrast, IM and SC injection of 0.5 μg LT-Gly33Asp or 0.5 μg LT/GM1 elicited no visible sign of an inflammatory response that was different from injection of the PBS vehicle control. LT-Gly33Asp and LT/GM1 are not reactogenic when administered by routes commonly used for parenteral vaccination.

### Example 5

### Toxin Binding to GM1 Ganglioside Receptors in Vivo Elicits Mediates Toxicity

To further point out the role of in vivo GM1 ganglioside binding as the underlying mechanism of LT-mediated toxicity, GM2/GD2 synthase knock out mice were studied (Takamya et al, 1996). These mice are unable to synthesize complex gangliosides including GM1 and, therefore, lack high affinity receptors. Intradermal injection of 0.5 μg wild type LT in the homozygous knock out mouse did not cause skin nodules to develop (Table 4, group 2), while injection of the same LT dose into heterozygous littermates produced an inflammatory response that was apparent by the formation of an inflammatory nodule at the site of injection (Table 4, group 1). A biopsy of the injection sites was taken and the tissue examined by histological methods. Histological examination of the injection site from the knockout mouse (no GM1 ganglioside) showed an absence of inflammatory cells or edema. Examination of tissues from the heterozygous littermates (normal GM1 ganglioside expression) showed injection site limited edema and inflammation following injection with wild type LT. These results are consistent that in vivo toxicity of LT is mediated through binding to the GM1 gangloside receptor.

TABLE 4
Intradermal injection of LT in GM2/GD2 synthase knock-out mice lacking
GM1

| Group | | Skin Nodules (cm) |
|---|---|---|
| 1 | GM2/GD2 synthase heterozygous | 1.2 ± 0.1 |
| 2 | GM2/GD2 synthase knock out | 0.18 ± 0.15 |

Mice received an intradermal injection of 25 μl PBS mixed with 0.5 μg LT. The next day swelling of the skin was measured. Data shown are means ± SD of 4 mice per group.

### Example 6

### Attenuation of the Toxicity of Other AB5 Toxins by Blocking High Affinity Receptor Binding

Cholera toxin (CT) is an example of another AB5 exotoxin, which is related to LT. CT shares about 80% amino acid identity with LT and the holotoxin is constructed of a single A-subunit, which is non-covalently associated with five B-subunits. GM1 ganglioside is also the natural high affinity receptor for CT. Intradermal or intramuscular injection of 0.5 μg CT caused skin nodules and muscle swelling, similar to LT. In contrast, injection of CT/GM1 complex did not produce an inflammatory response. These results confirm that disrupting in vivo binding to GM1 ganglioside receptors is a generally affective way to attenuate the toxicity of AB5 toxins. Toxic effects of other types of adjuvants may be eliminated by in vitro binding to receptor molecules (e.g., Toll-like receptors 2 and 4 binding LPS derivatives, TNFR family binding TNF-alpha).

### Example 7

### Cell Intoxication is Mediated by Toxin Binding to Cell Surface GM1 Gangliosides

In addition to in vivo evaluations, a rapid in vitro cell culture based assay was used to investigate the cytotoxicity of AB5 toxins. For example, cultured Y-1 adrenal cells were incubated with various amounts of LT or LT-Gly33Asp. Binding of exotoxin to cell surface exposed GM1 ganglioside receptors, uptake into the cell, stimulation of ADP-ribosylation activity, cAMP accumulation and subsequent disruption of microtubule stability, causes these cell to round up and release from the plastic substrate. The percentage of rounded Y1 cells is determined by microscopic examination of the cultures and is commonly used as a read out for LT and CT cytotoxicity. LT caused rounding in 70-100% of cultured cells within 8 hours at a concentration of 0.8 ng/ml, while no rounding was observed with LT-Gly33Asp at any concentration up to 100 ng/ml. The results are shown below in Table 5. Taken together, the in vivo toxicity and in vitro cytotoxicity of LT and CT is mediated by binding to GM1 ganglioside receptors on the cell. Blocking toxin binding to this receptor attenuates toxicity. AB5 toxin binding can be achieved by generation of mutations that interfere with receptor binding and by the use of small molecules that block the receptor-binding domain in the B-subunit.

TABLE 5
Evaluation of LT toxicity in Y-1 adrenal cells.

| | ng/ml | | | | | | | | | | | |
|---|---|---|---|---|---|---|---|---|---|---|---|---|
| | 100 | 50 | 25 | 12.5 | 6.3 | 3.1 | 1.6 | 0.8 | 0.4 | 0.2 | 0.1 | 0.05 |
| LT | +++ | +++ | +++ | +++ | +++ | +++ | +++ | +++ | ++ | + | −−− | −−− |
| LT-Gly33Asp | −−− | −−− | −−− | −−− | −−− | −−− | −−− | −−− | −−− | −−−− | −−− | −−− |

Confluent Y-1 adrenal cell cultures were incubated with various concentrations of LT or LT-Gly33Asp for 6-8 h at 37° C. Then the percentage of rounded cells were determined under the microscope, +++ 70-100% rounded cells, ++ 50-75%, + 25-50%, −−− <25%.

### Example 8

A recent study investigated the effects of in vitro coupling of CT to GM1 before application, on the skin on the immunogenicity of CT. However, the adjuvant activity towards co-administered antigens was not studied (Beignon et al., 2001). GM-1 binding deficient exotoxins would become especially attractive when shown that the adjuvant properties can be independent of tissue inflammation or other toxicities. However, several studies with LT mutants lacking GM1 binding capacity also lacked significant immunomodulatory activity (Aman et al, 2001; Nashar et al, 1996).

Transcutaneous Immunization (TCI) with Liquid Formulated LT/GM1 Adjuvanted Tetanus Toxoid on Intact Skin.

Unexpectedly, we now demonstrate that transcutaneous and parenteral administered LT/GM1 or LT-Gly33Asp has substantial adjuvant activity towards co-administered antigens while the immune response towards the adjuvant itself (i.e., immunogenicity) is reduced. For example, C57B1/6 mice were topically immunized by applying tetanus toxoid (TT) with or without 10 μg LT or LT-GM1 complex directly to shaven intact skin. After 45 minutes, the skin was rinsed with water to remove excess vaccine and adjuvant. Mice were immunized by the same method with three doses administered every two weeks, serum samples were collected two weeks after the third dose and analyzed for TT-specific antibodies. Mice immunized with TT alone (no adjuvant) generated low antibody titers (group 3, geometric mean 182 EU), while mice immunized with the same amount of TT adjuvanted with 10 μg of LT or LT-GM1 generated very high titer anti-TT antibody titers. The geometric mean titers (GMT) were 179,646 in the group immunized with LT and 57,710 in the group immunized with LT-GM1 adjuvanted vaccine (Table 6). These results demonstrate that LT and LT-GM1 were equally potent adjuvants when administered topically on intact skin. The adjuvant was required in order to generate a significant immune response to the co-administered antigen, TT.

TABLE 6
Serum IgG to Tetanus Toxoid (TT) after transcutaneous immunization (TCI)
with LT or LT-GM1

| | | | | | Assay values 1-10 | | | | | |
|---|---|---|---|---|---|---|---|---|---|---|
| | | | | | (Elisa Units) | | | | | |
| | | | | | Individual Mice | | | | | |
| | antigen | adjuvant | cofactor | | 1 | 2 | 3 | 4 | 5 | Geomertic |
| Groups | (Lf) | (μg) | (μg) | detecting | 6 | 7 | 8 | 9 | 10 | mean |
| 1 | TT (10) | LT (10) | — | TT IgG | 204102 | 146225 | 183724 | 323948 | 202170 | 179646 |
| | | | | | 185486 | 85832 | 163652 | 208284 | nsa | |
| 2 | TT (10) | LT (10) | GM1 (5) | TT IgG | 75059 | 58546 | 21953 | 17492 | 343311 | 57710 |
| | | | | | 25055 | 77573 | 109287 | nsa | nsa | |
| 3 | TT (10) | — | — | TT IgG | 44 | 7 | 18 | 32 | 9385 | 182 |
| | | | | | 3072 | 15 | 2193 | 1334 | nsa | |

Fifty μl of PBS containing 10 Lf units of TT alone or mixed with 10 μg of LT or LT-GM1 were applied to the shaven, hydrated back of the mouse. After 45 minutes, the back was washed with warm water. Two weeks post the last immunization serum antibody titers were determined by ELISA;
nsa, no serum available.
Data shown represent serum IgG against tetanus toxoid after three immunizations.

### Example 9

### Transcutaneous Immunization with LTGly33Asp Adjuvant

Using the same study design described in Example 8, groups of 9-10 mice were topically immunized with TT and 10 μg LT or the non-GM1 ganglioside-binding mutant, LT-Gly33Asp. Mice topically immunized with TT alone generated low titers anti-TT IgG titers (GMT=187) while, in contrast, the groups immunized with TT adjuvanted with either LT-Gly33Asp or wild type LT generated high titer anti-TT antibodies (GMT=62,817 and 53,177, respectively), indicting wild type and Gly33Asp LT are equipotent adjuvants (Table 7).

TABLE 7
Serum IgG to Tetanus Toxoid (TT) after transcutaneous immunization (TCI)
with LT or LT-Gly33Asp

| | | | | Assay values 1-10 | | | | | |
|---|---|---|---|---|---|---|---|---|---|
| | | | | (Elisa Units) | | | | | |
| | | | | Individual Mice | | | | | |
| | antigen | adjuvant | | 1 | 2 | 3 | 4 | 5 | Geometric |
| Groups | (Lf) | (μg) | detecting | 6 | 7 | 8 | 9 | 10 | mean |
| 1 | TT (10) | LT (10) | TT IgG | 83436 | 63293 | 50116 | 121818 | 176340 | 53177 |
| | | | | 114780 | 131805 | 6246 | 11745 | 31797 | |
| 2 | TT (10) | LT-Gly33Asp | TT IgG | 40458 | 91175 | 87899 | 95561 | 74721 | 62817 |
| | | (10) | | 40067 | 88870 | 5229 | 256217 | 86429 | |
| 3 | TT (10) | — | TT IgG | 87 | 67 | 886 | 28333 | 140 | 187 |
| | | | | 49 | 71 | 88 | 44 | nsa | |

Fifty μl of PBS containing 10 Lf units of TT alone or mixed with 10 μg adjuvant, LT or LT-Gly33Asp, were applied to the shaven, hydrated back of the mouse. After 45 minutes, the back was washed with warm water. Two weeks post the last immunization serum antibody titers were determined by ELISA.
Data shown represent serum IgG against tetanus toxoid after three immunizations.

### Example 10

### Disruption of the Stratum Corneum Aids the Topical Delivery of Antigens and Adjuvants into Skin

An important factor in the use of GM-1 binding deficient exotoxins in transcutaneous immunization is skin pretreatment. Previous studies demonstrated enhanced immunization via barrier-disrupted skin (Seo et al., 2000; U.S. Pat. No. 5,464,386). We have found that topical delivery of certain macromolecules and viral particles is improved by disruption of the outer skin layer (i.e., stratum corneum) for successful delivery into the skin (Guebre-Xabier et al, 2003). For example, application of influenza antigen and LT to hydrated skin of mice failed to induce significant antibody responses against influenza. However, when the skin was pretreated with 10 strokes with emery paper, the LT/influenza formulation was effectively delivered and high titer antibodies to influenza were generated (LT/Flu on sandpapered skin, group 2 geometric mean 11,172 vs LT/Flu on hydrated skin, group 1 geometric mean 116) (Table 8).

TABLE 8
Serum IgG to Influenza (Flu A) after transcutaneous immunization (TCI)

| | | | | | Assay values 1-5 | | | | | |
|---|---|---|---|---|---|---|---|---|---|---|
| | | | | | (Elisa Units) | | | | | |
| | antigen | adjuvant | | | Individual Mice | | | | | Geometric |
| Groups | (μg) | (μg) | pretreatment | detecting | 1 | 2 | 3 | 4 | 5 | mean |
| 1 | FluA (25) | LT (10) | hydration | Flu A IgG | 150 | 102 | 47 | 45 | 642 | 116 |
| 2 | FluA (25) | LT (10) | sandpaper | Flu A IgG | 15498 | 3137 | 6852 | 18563 | 28151 | 11172 |

C57B1/6 mice were immunized topically with influenza antigen mixed with LT. The skin was pretreated by hydration only or hydration + 10 strokes of sandpaper. Two weeks after the third immunization, serum samples were collected and the serum antibody titers against influenza were determined by ELISA.

Tape stripping or mild abrasion by rubbing with emery paper is a safe technique used clinically to remove the stratum corneum, and proven by us to be effective in delivery of complex antigens. Other skin pretreatment methods to enhance delivery include the use of microneedles, laser ablation or other physical and chemical penetration enhancement techniques (for example, U.S. Pat. Nos. 3,964,482 and 5,879,326; WO99/43350). The enhancing effect of skin pretreatment on the adjuvant activity of exotoxins may result in a reduction in the dose needed for induction of adequate immune responses.

### Example 11

### Attenuated LT/GM1 is a Potent Adjuvant but Poorly Immunogenic when Administered Topically with Skin Pretreatment

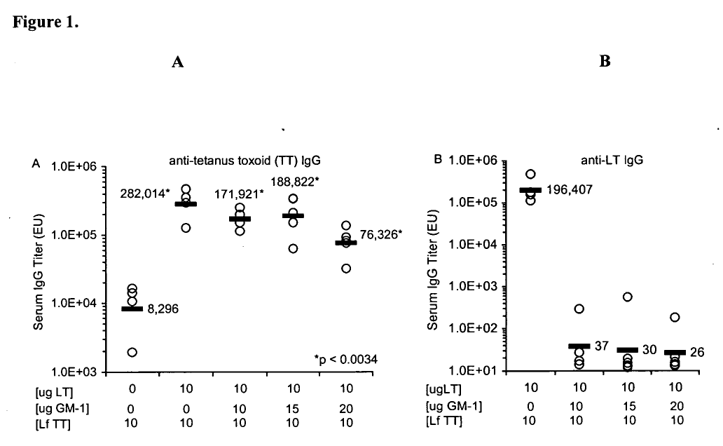

Various methods can be used to topically administer vaccine and adjuvant to the skin. As described in Examples 8 and 9, topical vaccination on intact skin in significantly improved by co-administering the adjuvant with a bystander antigen. Alternatively, the skin may be pretreated with a mild abrasive (emery paper or an abrasive pad) or tape stripped with the intent of disrupting or removing the stratum corneum. The stratum corneum functions as a protective barrier obstructing the entry of pathogens and environmental allergens from entering the body. The stratum corneum may be disrupted to improve topical delivery of large protein vaccines and antigens. The skin is briefly hydrated with saline, phosphate buffered saline or glycerol as examples followed by treatment with an abrasive or tape stripping to disrupt the outer barrier. The simplest method is to apply a liquid solution containing the vaccine and LT adjuvant directly to the pretreated skin. An example of this procedure is illustrated in FIG. 1. In this study, mice were shaved near the base of the tail one day before immunization. The shaven skin was tape stripped (10 times) to remove the stratum corneum. Tetanus toxoid (10 Lf) alone or admixed with 10 μg LT or with 10 μg of LT/GM-1 (10 μg, 15 μg and 20 μg GM-1) was applied directly to the pretreated skin for 1 hour. The skin was thoroughly washed to remove excess TT and LT. All groups were immunized with 3-doses (day 1, 15 and 29) and serum was collected two weeks after the last dose. The results in FIG. 1A show the serum antibody titers to TT for each group. The group immunized with TT alone generated relatively low titer antibodies (GMT=8,230). The group immunized with LT adjuvanted TT generated anti-TT IgG titers that were 34-fold greater (GMT=282,000) than the non-adjuvanted group. In addition, mice immunized with LT/GM-1 attenuated adjuvant and vaccine, also generated significantly higher (9 to 20-fold) anti-TT IgG titers (GMT=76,000 to 189,000) compared to the group immunized without adjuvant. Serum antibodies to LT were also examined and the results are depicted in FIG. 1B. Mice topically immunized with LT developed high antibody titers to LT (GMT=196,000), while mice receiving LT/GM-1 complex had little or no anti-LT antibody titers (GMT=26-37). These results demonstrate that the attenuated LT/GM1 adjuvant was equally potent as LT and was poorly immunogenic when administered on skin pretreated to disrupt the stratum corneum.

### Example 12

### Transcutaneous Immunization with Attenuated AB5 Adjuvants and Antigens Delivered with a Patch

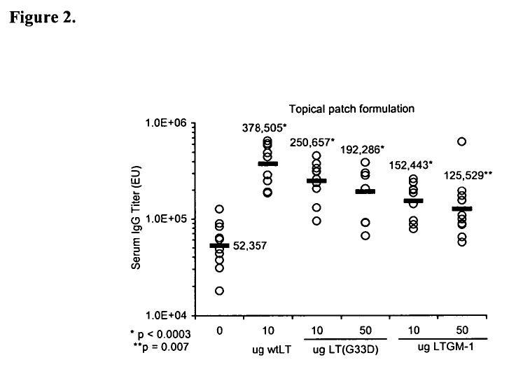

Another method for topical administration of an antigen and adjuvant is by the use of a patch. FIG. 2 illustrates the use of a patch to deliver TT vaccine adjuvanted with LT, LTGly33Asp or LT/GM1. In this study, mice were shaved and the skin pretreated with emery paper. Patches were constructed of a 1 cm² gauze pad affixed to an adhesive backing. The gauze was loaded with 10 Lf of TT alone or admixed with LT (10 μg), LTGly33Asp (10 μg and 50 μg) or LT/GM1 (10 μg and 50 μg). These patches were applied for 1 hour, removed and the skin rinsed. All animals were immunized twice (day 1 and 15) and serum collected weeks after the second dose. The results in FIG. 2 show that LT, LTGly33Asp and LT/GM1 significantly enhanced (p≦0.007) the immune response to co-administered TT. At the 10 μg dose, LT/GM1 was less adjuvanting than LT (p=0.001) or LTGly33Asp (p=0.034). LT and LTGly33Asp were equipotent adjuvants (p=0.07) when administered in a patch with a vaccine antigen.

Immune responses produced by the adjuvant-antigen formulations may include the eliciting antigen-specific antibodies and cytotoxic lymphocytes (CTL). Antibody can be detected by immunoassay techniques or functional neutralizing assays. In an in vitro immunoassay, serial dilutions of sera or other body fluids are incubated with antigen after which the antigen-bound antibody is detected by labeling with fluorochromes. In neutralization assays, serial dilutions of sera (or other body fluids) are investigated for their potential to block a specific cellular response, such as antigen-mediated signal transduction, protein production, toxicity, or infectivity with specific pathogens. Specific CTL can be detected in vitro by proliferation and/or cytokine secretion assays. In proliferation assays, T cells are incubated with the antigen after which proliferation of these cells is measured by radioactive thymidine incorporation. Cytokine secretion assays involve stimulation of T cells with antigen followed by detection of intra- and/or extracellular concentrations of cytokines such as interferon-gamma, interleukin-2, -4, -5, -10, or -12.

### Example 13

### Transcutaneous Immunization with LT-Gly33Asp Adjuvant Potentiates Antigen-Specific Cellular Immune Responses

In addition to antibody responses, some infections are controlled by cell-mediated immune responses. Previous reports showed the capacity of LT to induce both cellular as well as humoral immune response towards co-administered antigens (Hammond et al., 2001; Yu et al., 2002; Guebre-Xabier et al., 2003). We investigated whether GM1-binding deficient LT formulations were also capable of inducing cellular immune responses. For example, mice were topically immunized on the back with TT alone, or combined with LT or LTGly33Asp. Three weeks after three rounds of immunization (day 1, 15 and 29), the draining inguinal lymph nodes of each experimental group were isolated, pooled, and converted into a cell suspension by gentle rubbing through nylon gauze mesh. The cells were cultured over night in the presence or absence of TT, and cytokine secreting lymphocytes were detected by ELISPOT. We found that animals immunized with LT or LTGly33Asp adjuvanted TT vaccine generated cellular immune responses to TT. As can be seen in Table 9 below, lymphocytes recovered from lymph nodes of mice immunized with TT alone contained few IFNγ (13 spots/10⁶ cells) and IL4 (36 spots/10⁶ cells) producing lymphocytes. In contrast, lymph nodes recovered from mice immunized with LT adjuvanted vaccines contained 10 times more IFNγ (136 spots/10⁶ cells) and IL4 (223 spots/10⁶ cells) producing lymphocytes compared to immunization with TT alone. Likewise, lymph nodes recovered from mice immunized with LTGly33Asp adjuvanted vaccine contained about 10 times more IFNγ (131 spots/10⁶ cells) and IL4 (281 spots/10⁶ cells) producing lymphocytes (Table 9) than the non-adjuvanted group. These results clearly show that both cellular immune responses and the humoral antibody responses to a bystander antigen are significantly amplified by co-administering LT or LTGly33Asp. Furthermore, the mutant LT was found to be equally potent as the wild type LT, without the toxicity (Table 3) of wild type LT.

TABLE 9
Tetanus Toxoid (TT)-specific immune cells in the inguinal draining lymph
nodes from TCI-immunized mice secrete IFN-γ and IL4

| | antigen | adjuvant | | Assay Values |
|---|---|---|---|---|
| Groups | (Lf) | (μg) | detecting | Spots/10⁶ cells |
| 1 | TT (10) | LT (10) | IFN | 136 |
| | | | IL4 | 223 |
| 2 | TT (10) | LTGly33Asp (10) | IFN | 131 |
| | | | IL4 | 281 |
| 3 | TT (10) | — | IFN | 13 |
| | | | IL4 | 36 |

Three weeks after three immunizations, the inguinal lymph nodes for each group were collected, pooled, and cells were cultured in the presence of TT. Spots indicating the presence of IFNγ or IL4-secreting cells were enumerated by dissecting microscope (ELISPOT). Data shown represent the number of TT-specific immune cells per 10⁶ cells.

### Example 14

### Transcutaneous Immunization with GM1 Non-Binding Toxins Potentiate the Cellular Immune Response to Poorly Immunogenic Bystander Antigens

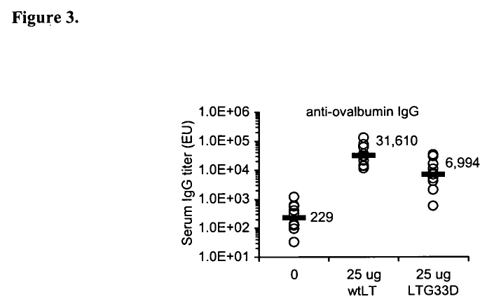

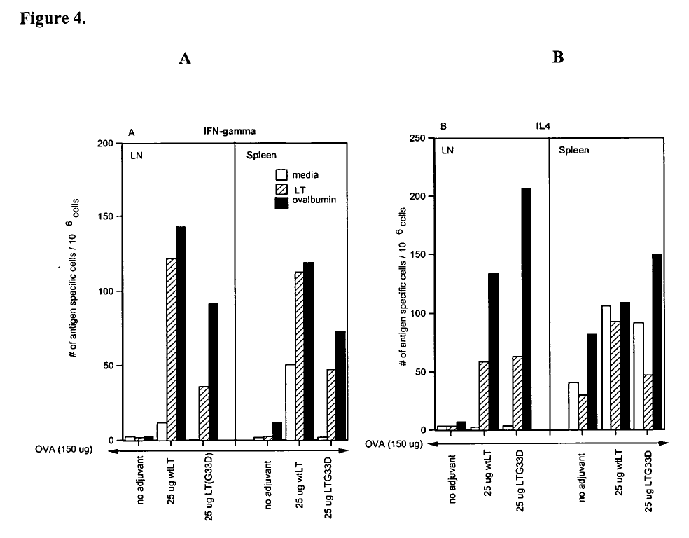

The immune response to poorly immunogenic antigens such as ovalbumin (OVA) may be significantly improved by the use of an adjuvant. The humoral and cellular immune responses elicited by topical immunization with OVA adjuvanted with LT and LTGly33Asp was compared. In this study, mice were prepared for topical immunization by shaving and pretreating saline hydrated skin with emery paper to disrupt the stratum corneum. Patches constructed of 1 cm² gauze pad affixed to an adhesive backing were loaded with 150 μg OVA alone or admixed with 25 μg LT or LTGly33Asp. Patches were worn overnight, removed and the skin rinsed with water. Groups of 10 mice were immunized with three doses (day 1, 15 and 29) and serum, inguinal lymph nodes and spleens were collected two weeks after the third immunization. As depicted in FIG. 3, mice immunized with LT and LTGly33Asp adjuvanted OVA generated antibody titers that were significantly greater (GMT=32,000 and 7,000, respectively) than the group immunized with OVA alone (GMT=229). ELISPOT analysis was used to characterize the cellular immune response to topical immunization. The results in FIGS. 4A and 4B show the proportion of lymph node (LN) and spleen cells stimulated to produce IFN-γ and IL-4 when cultured overnight with OVA or LT. LNs recovered from animals immunized with OVA alone (no adjuvant) were devoid of cells responding to re-stimulation with OVA or LT. In contrast, LN recovered from mice immunized with OVA adjuvanted with either LT or LTGly33Asp did respond to in vitro re-stimulation with OVA or LT by producing IFN-γ and IL-4. Spleen cells recovered from immunized mice were also characterized for response to OVA and LT. FIG. 4A shows spleen cells recovered from mice immunized with OVA and no adjuvant contained very few or no IFN-γ producing lymphocytes. In contrast, spleens from mice topically immunized with OVA adjuvanted with LT or LTGly33Asp did respond to re-stimulation by producing IFN-γ. Due to a high background, IL-4 production by splenocytes could not be conclusively interrupted. These studies demonstrated the essential role of LT-adjuvants in the generation of immune responses to poorly immunogenic antigens delivered by the topical route. Non-GM1 ganglioside receptor binding LTGly33Asp did elicit OVA specific antibody and cellular immune responses that were comparable to the wild type LT adjuvant. This example is significant since it illustrates the advantages of using the non-reactogenic mutant adjuvant to stimulate both humoral and cellular immune responses directed towards poorly immunogenic vaccines.

### Example 15

### Non-GM1 Ganglioside Receptor Binding AB5 Toxins can be Used to Adjuvant Parenteral Injected Vaccines and Antigens

The reactogenicity of LT and CT has limited their use as oral, nasal or parenteral (for example, subcutaneous, intradermal, and intramuscular) injected adjuvants in humans. The following examples demonstrate that non-GM1 ganglioside binding AB5 toxins can be used to stimulate immune responses to bystander antigens when administered by injection without toxicity. In this example (Table 10), groups of 10 mice were intradermally injected with TT alone (group 5) or with soluble GM1 ganglioside control (group 4). Separate groups were immunized with TT adjuvanted with 0.5 μg of LT, LT-Gly33Asp or LT-GM1 (groups 1, 2 and 3, respectively). Two weeks after two rounds of immunization (day 1 and 15), serum samples were collected and analyzed for antibody titers to TT. Mice immunized with TT alone or admixed with soluble GM1 generated moderate anti-TT titers (GMT=13,010 and 20,019, respectively). This is contrasted to the very high increase (9 to 16-fold) in antibody titers generated by mice immunized with TT adjuvanted with LT (GMT=204,271), LTGly33Asp (GMT=112,842) or LT/GM1 (GMT=146,794). Examination of the injection sites showed that only the group injected with wild type LT developed edema and inflammation at the site of injection as previously described (Tables 2 and 3). Similar results were obtained when the antigen and adjuvant were administered by the intramuscular and subcutaneous routes. These results show that LTGly33Asp and LT/GM1 are potent adjuvants comparable to wild type LT. Unlike wild type LT, however, these attenuated adjuvants can also be administered by parenteral injection without causing local reactogenicity or systemic toxicity.

TABLE 10
Serum IgG to tetanus toxoid (TT) after intradermal immunization with LT,
LT-Gly33Asp, or LT-GM1.

| | | | | Assay values 1-10 | | | | | |
|---|---|---|---|---|---|---|---|---|---|
| | | | | (Elisa Units) | | | | | |
| | | adjuvant | | Individual Mice | | | | | |
| | antigen | or cofactor | | 1 | 2 | 3 | 4 | 5 | Geometric |
| Groups | (Lf) | (μg) | detecting | 6 | 7 | 8 | 9 | 10 | mean |
| 1 | TT (0.01) | LT (0.5) | TT IgG | 122765 | 138671 | 78521 | 182431 | 378770 | 204271 |
| | | | | 465820 | 239767 | 153676 | 352020 | 226657 | |
| 2 | TT (0.01) | LT-Gly33Asp | TT IgG | 171451 | 89280 | 94985 | 123969 | 108867 | 112842 |
| | | (0.5) | | 91830 | 103277 | 67078 | 269941 | 99338 | |
| 3 | TT (0.01) | LT (0.5) + | TT IgG | 141182 | 86535 | 146028 | 150863 | 120443 | 146794 |
| | | GM1 (0.25) | | 202250 | 204134 | 185446 | 120203 | 155735 | |
| 4 | TT (0.01) | GM1 (0.25) | TT IgG | 12720 | 37868 | 12063 | 21612 | 28277 | 20019 |
| | | | | 11094 | 18919 | 16826 | 27529 | 29941 | |
| 5 | TT (0.01) | — | TT IgG | 2378 | 39743 | 26603 | 8307 | 13146 | 13010 |
| | | | | 9436 | 12522 | 15869 | 33684 | 8010 | |

Mice received an intradermal injection of 25 μl PBS containing 0.01 Lf units of TT alone or mixed with 0.5 μg adjuvant, LT, LT-Gly33Asp or LT-GM1, at the back of the mouse. Two weeks after the last immunization, serum antibody titers were determined by the ELISA method.
Data shown represent serum IgG against tetanus toxoid after two immunizations.

### Example 16

### Non-GM1 Ganglioside Binding AB5 Adjuvants can be Used to Stimulate Immune Responses to Parenteral Injected Vaccines and Antigens

Application of LTGly33Asp as a parenteral injected adjuvant was further evaluated with other antigens. For example, groups of mice were intradermal injected with inactivated influenza vaccine with or without LTGly33Asp. Two weeks after two rounds of immunizations (day 1 and 15), serum samples were collected and analyzed for antibodies to influenza antigens using an ELISA method. Mice immunized with influenza vaccine alone developed low titer antibodies (GMT=761) while mice immunized with the mutant LT adjuvanted vaccine developed antibody titers (GMT=34,540) that were 45-fold higher than without the adjuvant (Table 11). LTGly33Asp can be generally used as an adjuvant to stimulate immune responses to bystander vaccines and antigens. Non-GM1 ganglioside binding AB5 toxins are superior to the wild type AB5, since these adjuvants are not inflammatory when injected into tissues.

### Example 17

### Use of Non-GM1 Binding AB5 Toxins with Vaccines Injected by Intramuscular and Subcutaneous Routes

The previous example shows that attenuated AB5 potentiate immune responses to co-administered antigens when injected intradermal. Since LTGly33Asp and LT/GM1 are not inflammatory or reactogenic when injected by IM or SC routes (Example 4), these adjuvants may also be used with vaccines and antigens administered by different routes. For example, mice were intramuscular (im) injected (thigh muscle) or subcutaneous (sc) injected with TT alone or adjuvanted with LTGly33Asp. Two weeks after two rounds of immunization (day 1 and 15), serum samples were collected and analyzed for TT-specific antibody titers. The results in Table 12 show that IM injection with a low dose (0.01 Lf) of TT elicited low titer (GMT=131) antibodies to TT, while adjuvanting the vaccine with 0.5 μg LTGly33Asp elicited a very significant 277-fold increase in antibody titer (GMT=36,250). Mice immunized by the SC route, responded poorly to TT (GMT<10) while co-administering TT with LT-Gly33Asp elicited a 6,700-fold increase in antibody titer (GMT=40,154). LTGly33Asp is an effective adjuvant when administered by different parenteral routes of injecting (ID, IM and SC). In contrast to wild type LT, the mutant LT was not inflammatory when administered by any of these routes.

TABLE 11
Serum IgG to recombinant protective antigen from Bacillus anthracis (rPA)
or Influenza antigen (Flu) after intradermal immunization with LT-Gly33Asp

| | | | | Assay values 1-5 | | | | | |
|---|---|---|---|---|---|---|---|---|---|
| | | | | (Elisa Units) | | | | | |
| | antigen | adjuvant | | Individual Mice | | | | | Geometric |
| Groups | (μg) | (μg) | detecting | 1 | 2 | 3 | 4 | 5 | mean |
| 1 | Flu (0.1) | — | Flu A IgG | 487 | 619 | 292 | 2585 | 1120 | 761 |
| 2 | Flu (0.1) | LT-Gly33Asp (0.5) | Flu A IgG | 27328 | 13324 | 74772 | 41288 | 43735 | 34540 |

Mice received an intradermal injection of 25 μl PBS containing 0.1 μg Flu (trivalent split virus antigen), alone or mixed with 0.5 ug LTGly33Asp at the back of the mouse. Two weeks post the last immunization serum antibody titers were determined by ELISA.
Data shown represent serum IgG against rPA or Flu A after two immunizations.

TABLE 12
Serum IgG to tetanus toxoid (TT) after intramuscular or subcutaneous
immunization with LT-Gly33Asp

| | | | | | Assay values 1-5 | | | | | |
|---|---|---|---|---|---|---|---|---|---|---|
| | | | | | (Elisa Units) | | | | | |
| | antigen | adjuvant | | | Individual Mice | | | | | Geometric |
| Groups | (Lf) | (μg) | route | detecting | 1 | 2 | 3 | 4 | 5 | mean |
| 1 | TT (0.01) | — | im | TT IgG | 17 | 22 | 15 | 3086 | 2200 | 131 |
| 2 | TT (0.01) | LT-Gly33Asp | im | TT IgG | 27043 | 31175 | 52400 | 39738 | 35655 | 36250 |
| | | (0.5) | | | | | | | | |
| 3 | TT (0.01) | — | sc | TT IgG | 1 | 1 | 23 | 464 | 1 | 6 |
| 4 | TT (0.01) | LT-Gly33Asp | sc | TT IgG | 43510 | 50691 | 42889 | 17876 | 61734 | 40154 |
| | | (0.5) | | | | | | | | |

Mice received an intramuscular or subcutaneous injection of 25 μl PBS containing 0.01 Lf units of TT alone or mixed with 0.5 μg LT-Gly33Asp. Two weeks post the last immunization serum antibody titers were determined by ELISA.
Data shown represent serum IgG against TT after two immunizations.

### Example 18

### Use of Non-GM1 Ganglioside Cholera Toxin (CT) as a Non-Toxic Adjuvant

Cholera toxin (CT) is an AB5 exotoxin, which shares about 80% amino acid identity with LT. The adjuvanticity of other exotoxins detoxified by B-subunit modification was also examined. For example, mice were immunized by topical or by parenteral injection (ID and IM) with TT alone or admixed with CT or CT/GM1 complex. Two weeks after two rounds of immunization (day 1 and 15), serum samples were collected and analyzed for TT-specific antibody titers. In Table 13 below, TT elicited low titer antibodies when administered alone by ID injection (group 1, GMT=116), IM injection (group 4, GMT=201) or by the topical route (group 7, GMT=1,104). TT adjuvanted with CT or CT/GM1 and injected ID produced titers that were 823 to 435-fold higher (groups 2 and 3, respectively) than the non-adjuvanted vaccine. TT adjuvanted with CT or CT/GM1 and injected in mice by the IM route produced antibody titers to TT that were 426- to 272-fold higher (groups 5 and 6, respectively) than the non-adjuvanted vaccine. TT adjuvanted with CT or CT/GM1, and administered topically to mice, developed antibody titers to TT that were 105- to 30-fold greater (groups 8 and 9) than the non-adjuvanted vaccine. Unlike CT, CT/GM1 was not inflammatory when administered by ID or IM injection. Furthermore, CT was only slightly more active (1.6 to 3-fold) than CT/GM1. These results demonstrate that preparations of other AB5 toxins that do not bind the GM1 ganglioside receptor in vivo also can be use to stimulate immune responses to bystander antigens without toxic side effects. A mutant CTGly33Asp holotoxin is expected to have the same properties as CT/GM1, LT/GM1, LTArg192Gly/GM1 (see Example 27) and LTGly33Asp.

TABLE 13
Serum IgG to tetanus toxoid (TT) after parenteral or topical immunization
with CT or CT-GM1

| | | | | | Assay values 1-5 | | | | | |
|---|---|---|---|---|---|---|---|---|---|---|
| | | | | | (Elisa Units) | | | | | |
| | antigen | adjuvant | | | Individual Mice | | | | | Geometric |
| Groups | (Lf) | (μg) | route | detecting | 1 | 2 | 3 | 4 | 5 | mean |
| 1 | TT (0.01) | — | id | TT IgG | 98 | 216 | 1569 | 6 | 107 | 116 |
| 2 | TT (0.01) | CT (0.5) | id | TT IgG | 49754 | 228291 | 66812 | 129812 | 80294 | 95419 |
| 3 | TT (0.01) | CT-GM1 | id | TT IgG | 38868 | 85050 | 65498 | 50482 | 29897 | 50449 |
| 4 | TT (0.01) | — | im | TT IgG | 1085 | 23955 | 12 | 150 | 7 | 201 |
| 5 | TT (0.01) | CT (0.5) | im | TT IgG | 71077 | 113082 | 75238 | 123721 | 61438 | 85603 |
| 6 | TT (0.01) | CT-GM1 | Im | TT IgG | 46043 | 36864 | 45759 | 97140 | 64631 | 54653 |
| 7 | TT (10) | — | TCI | TT IgG | 3722 | 2305 | 2010 | 103 | 925 | 1104 |
| 8 | TT (10) | CT (25) | TCI | TT IgG | 11222 | 14300 | 29533 | 63092 | 125687 | 32734 |
| 9 | TT (10) | CT-GM1 | TCI | TT IgG | 18690 | 7510 | 14793 | 7480 | 9791 | 10874 |
| Mice received a parenteral (intradermal, id; intramuscular, im) injection or a topical (transcutaneous, TCI) application of 25 μl PBS containing 0.01 Lf units (parenteral) or 10 Lf units (topical) of TT. For parenteral application, TT was given alone or mixed with 0.5 μg CT or 0.5 μg CT + 0.25 μg GM1. For topical application, TT was given alone or mixed with 25 μg CT or | | | | | | | | | | |
| # 25 μg CT + 12.5 μg GM1. Two weeks post the last immunization serum antibody titers were determined by ELISA. | | | | | | | | | | |

Data shown represent serum IgG against TT after two immunizations.

### Example 19

### Use of Non-Toxic, Non-GM1 Ganglioside AB5 Toxins as Adjuvants to Potentiate Immune Responses to Tumor Associated Antigens for Treatment of Immunogenic Cancers

A great limitation to the development of effective therapeutic vaccines to treat cancers is the lack of potent adjuvants. Tumor associated antigens (TAA) are typically poor immunogens. In an attempt to improve vaccine potency, a number of strategies have been tried including the use of purified MHC epitopes, in vitro activation of a patients' dendritic cells pulsed with TAA, use of attenuated virus vectors and vaccines to stimulate immune responses. In addition to vaccines for infectious diseases, we have also investigated the use of LTGly33Asp as an adjuvant for use with therapeutic cancer vaccines. To illustrate this application, a mouse model was used to evaluate the efficacy of cancer vaccines and vaccination strategies using attenuated AB5 adjuvants. In this model, mice were first inoculated subcutaneously with MO5 cancer cells which were genetically modified to express the chicken protein OVA. Tumor bearing mice were then immunized with OVA. Vaccine efficacy is determined by measuring tumor size measured and monitoring survival over time. For example, C57B1/6 mice were injected subcutaneously with a small amount of MO5 cells. Three days later, the mice were immunized by intradermal injection with OVA alone or adjuvanted with LT-Gly33Asp. A booster immunization was administered ten days later. Over time, mice are monitored for outgrowth of subcutaneous tumors. Three weeks after tumor inoculation, visible tumor outgrowth (average tumor diameter 43 mm²) was observed in 50% of the non-OVA immunized mice (group 1, Table 14). Forty percent of mice immunized with OVA alone developed measurable tumors (group 2, average diameter 32 mm²). However, no tumors were found at this time in mice that were immunized with OVA adjuvanted with LTGly33Asp (group 3). As shown in Table 14, mice immunized with poorly immunogenic OVA alone, developed low titer anti-OVA IgG (GMT=255) and very few antigen specific, IFNγ inducible lymphocytes were detected in the lymph nodes (1 spot/10⁶ cells) and spleen (4 spot/10⁶ cells). This was in contrast to the group immunized with LTGly33Asp adjuvanted OVA where a large number of antigen inducible, IFNγ-producing lymphocytes were detected in the lymph nodes (426 spot/10⁶ cells) and spleen (378 spot/10⁶ cells). As a result, the enhanced immune responses were capable of destroying OVA expressing target cells and suppressing tumor growth. These results demonstrate the usefulness of GM1-binding deficient LT formulations as vaccine adjuvants in the field of cancer as well as infectious diseases.

TABLE 14
Induction of immune responses and tumor control by LT-Gly33Asp

| | | | | | Number of IFNγ | |
|---|---|---|---|---|---|---|
| | | | Number of | Antibody titer | secreting cells | |
| | Antigen | Adjuvant | tumor bearing | Geometric mean | (spots/10⁶ cells) | |
| Group | (μg) | (μg) | mice % | (EU) | Lymph nodes | spleen |
| 1 | — | LT-Gly33Asp (0.5) | 50 | 5 | 1 | 1 |
| 2 | OVA (150) | — | 40 | 255 | 1 | 4 |
| 3 | OVA (150) | LT-Gly33Asp (0.5) | 0 | 29416 | 426 | 378 |
| Mice were inoculated with 10⁵ OVA-expressing MO5 cells by subcutaneous injection at day 0. Three days later, mice were immunized by intradermal injection with 150 μg ovalbumin (OVA) and/or 0.5 μg LT-Gly33Asp followed by booster immunizations every 2 weeks. The number of mice bearing tumors was determined three weeks after tumor inoculation. Serum antibody titers against OVA were measured by Elisa | | | | | | |
| # two weeks post the last immunization. Inguinal lymph nodes and spleens were collected for each group, and cells were cultured in the presence of 5 μg/ml SIINFEKL, the immunodominant peptide of OVA. Spots indicating the presence of IFNγ-secreting cells were enumerated by dissecting microscope (ELISPOT). | | | | | | |

### Example 20

### Use of Non-Toxic, Non-GM1 Ganglioside AB5 Adjuvant Topically Delivered from a Patch to Potentiate Immune Responses to Injected Antigens

The Immune Stimulant (IS) patch is an adjuvant delivery system designed to improve the potency and efficacy of parenteral injected vaccines. IS-patches are formulated to be simple to apply over the injection site at the time of vaccination, similar to a Band-Aid. LT or CT are the active ingredients used in formulated IS-patches. In this application, LT is delivered directly to skin dendritic cells, Langerhans cells (LCs), located in the superficial layer of the epidermis. Preclinical studies and human clinical trials have demonstrated that LT activation of skin LCs, at the time of parenteral immunization, significantly potentiates antibody and cellular immune responses to injected antigens.

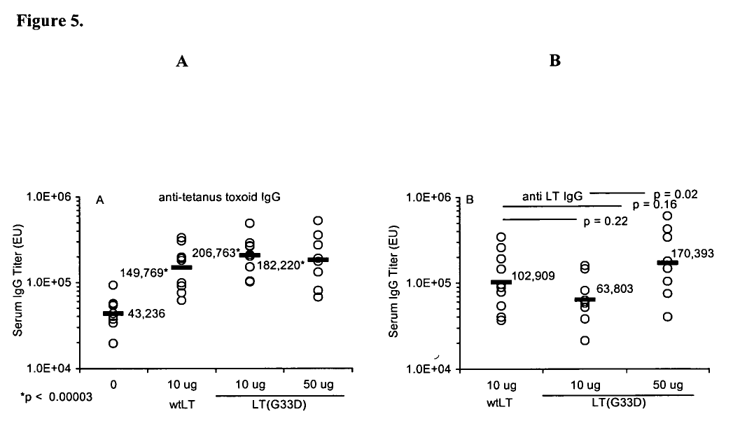

Although moderate and self-resolving, LT may be reactogenic when topically applied to skin pretreated to disrupt the stratum corneum. A study was conducted to assess the adjuvanting activity of LTGly33Asp when used topically to stimulate immune responses to an injected antigen. TT was used as a model antigen to assess this concept. Groups of mice (N=8-10/group) were prepared by shaving dorsal caudal one-day before immunization. Immediately before immunization, the shaven skin was saline hydrated and gently pretreated with emery paper to disrupt the stratum corneum. Tetanus toxoid (0.2 Lf) was ID injected into the pretreated skin and a 1 cm² gauze patch loaded with phosphate buffered saline (vehicle control) or with 10 μg of LT or with LTGly33Asp (10 μg or 50 μg) was applied over the site of injection. Patches were removed the next day and the skin was rinsed with water. Mice were immunized on study day 1 and 15 and serum was collected 2 weeks after the second immunization. The results in FIG. 5 show the group receiving an ID injection with TT with the placebo patch generated moderate anti-TT IgG titers (GMT=43,000). The group receiving ID injected TT with an LT IS-patches had a significantly higher (p≦0.00003) antibody titer (GMT=150,000). Likewise, the group receiving patches containing 10 μg LTGly33Asp also generated significantly higher (p≦0.00003) antibody titers to TT (GMT=207,000). Increasing the LTGly33Asp dose to 50 μg did not affect the antibody titer to TT (GMT=182,000) indicating that a 10 μg LTGly33Asp dose was at or above the maximal effective dose for stimulating an optimal immune response to TT.

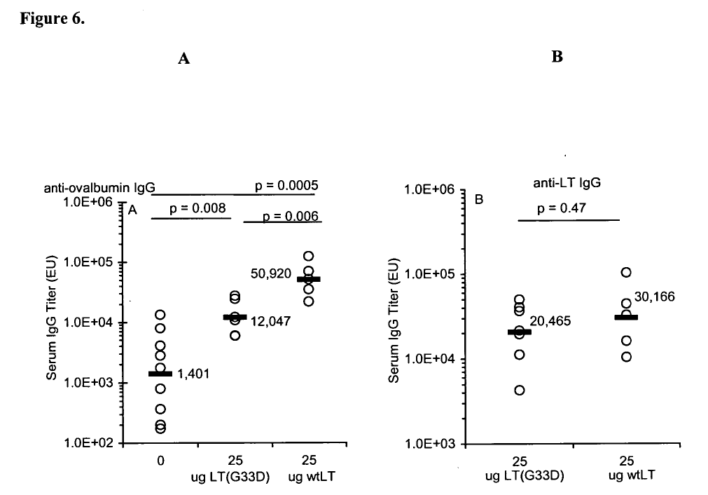

An advantage of the IS-patch is that it can be used to improve the immunogenicity of poorly immunogenic antigens without changing the vaccine formulation or the route of administration of the injected vaccine. OVA is an example of such an antigen. To illustrate, mice were prepared for immunization as described in FIG. 6. A high dose of OVA (150 μg) was intradermal injected into shaven pretreated skin. A 1 cm² gauze patch affixed to an adhesive backing was loaded with PBS (placebo control) or 25 μg of LT or LTGly33Asp was applied directly over the injection site. Patches were removed the next day and the skin rinsed. Groups of 5-9 mice were immunized with three doses (day 1, 15 and 29) and serum was collected two weeks after the third dose. The results in FIG. 6 show that three doses of OVA with a placebo patch elicited low titer antibodies (GMT=1,401). Anti-OVA titers were increased 10-fold (GMT=12,000) and 37-fold (GMT=51,000) in the groups receiving LTGly33Asp or LT IS-patches, respectively. LTGly33ASP and LT elicited comparable anti-LT IgG titers (GMT=20,000 and 30,000, respectively). The potency of poorly immunogenic antigens and vaccines can be significantly increased by topical application of LTGly33Asp (and LT/GM-1) at the time of injection of a vaccine.

Generation of Non-GM1 Ganglioside Binding AB5 Toxins by Mutagenesis, Chemical Derivatives and Receptor Blocking Antagonists

LT and CT Holotoxins (AB5 toxins) and their respective B-pentamers, EtxB and CtxB, mediate a profound affect on the immune response to bystander antigens as a result of high affinity binding to cell surface receptors. Both species of extoxin bind to the same receptor, GM-1 ganglioside (Spanger, 1992). Receptor binding is mediated through the interaction of the five B-subunits with the cell surface exposed receptor. The binding is high affinity for CT and CtxB (K_{D}=6×10⁻¹⁰ M) and for LT and EtxB (K_{D}=7×10⁻¹⁰ M). GM1 is ubiquitously expressed by mammalian cells and it is composed of a pentasacchride moiety, which is anchored in the plasma membrane through a ceramide tail. Unlike CT, LT also has reduced affinity for GD1b, asialo-GM-1, lactosylceramide and some galactoproteins (reviewed in Spanger, 1992). Extensive scientific literature argues that GM1 receptor binding is essential to immunostimulating activity. This is evident from numerous reports that demonstrate a loss of immune stimulating activity when GM1 ganglioside receptor binding is perturbed (Nasher, et al., 1996, Williams, et al., 1997, Nasher et al., 1997, Nasher et al., 2001, Bone, et al., 2002, Truitt, et al., 1998, and Jobling and Holms, 2002). The observation that non-receptor binding toxins, e.g., LTGly33Asp, are potent adjuvants and are non-reactogenic when used topically is an unexpected finding.

A number of strategies can be used to generate AB5 toxins, which do not bind, or bind with reduced affinity, to GM1 ganglioside on cells. The following are examples of methods that can be used to reduce affinity or prevent receptor binding. These strategies include, for example, 1) the use of random and site-specific mutagenesis to replace wild type amino acids by substitution of various residues within the GM1 ganglioside binding pocket; 2) mutagenesis to create amino acid substitutions outside or adjacent to the binding pocket with the intent to cause destabilizing conformational changes within the receptor pocket; 3) block GM1 ganglioside binding by introducing chemical modification to residues essential for receptor binding; 4) generation of receptor antagonists that block the pentasaccharide(OS)-GM1 binding site; and 5) generation of genetic fusion proteins (e.g., histidineₙ) that sterically block the GM1 binding pocket. The following examples describe methods that can be used to generate AB5 toxin variants that have reduced or no affinity for GM1 ganglioside, exhibit attenuated toxicity, and stimulate in vivo immune responses to bystander antigens. AB5 variants produced by these methods are expected to have characteristics similar to LTGly33Asp, LT/GM1, LTArg192Gly/GM1 (see Example 27) and CT/GM1.

### Example 21

### Receptor Pocket Mutagenesis

The crystalline structures of LT and CT have been determined. High-resolution analysis of the binding pockets of LT and CT show they are identical [13, 34]. The contributing amino acids are conserved with the exception of residue 13, which is a histidine in CT and may be either a histidine or arginine in LT. The three dimensional image of the CTB-pentamer and receptor complex have been solved for lactose (Sixman et al., 1992), galactose (Merritt et al., 1994), D-galactopyranosyl-β-D-thio-galatopyranoside and meta-nitrophenyl-D-galactopyranoside (Merritt et al., 1997). These studies identified more than 12 residues in CT and LT that interact directly with the OS-GM1 of GM1 ganglioside. These studies show that OS-GM1 binds within a pocket formed by Glu11, Tyr12, His13 (or Arg13), Asn14, Glu51, Gln56, His57, Gln61, Trp88, Asn90 and Lys91. Random and site directed mutagenesis have been used to identify those residues within the pocket that are essential to binding. Using this approach, each residue can be systematically substituted with a different amino acid and the effect of the change upon toxin binding to GM1 ganglioside or OS-GM1 determined using the GM1 ELISA method (De Hann, et al., 1996).

This approach has been used to identify residues that are not critical to binding as well as those that are essential to binding. For example, substitution of Glu at position 51 (Glu51) with Lys (Glu51Lys) or Lys91 with Asp (Lys91Asp) binds OS-GM1 with the same affinity as wild type CtxB indicating that positions 51 and 91 are less critical to receptor binding. In contrast, substitution of Tyr12 with Asp (Tyr12Asp) completely disrupts the interaction of the B-pentamer with its receptor (Jobling and Holms, 2002). Site directed mutagenesis has also been used to destabilize the binding pocket by making conservative substitutions in amino acids that are adjacent to the binding domain. For example, substitution of the Ala in position 95 with Asp (Ala95Asp) only slightly reduces binding receptor binding affinity. Therefore, a structure-function approach can be used to generate other mutant LT and CT (e.g., Tyr12Asp), which do not recognize the GM1 ganglioside on the cell surface. These mutants are expected to have the same biological properties as LTGly33Asp and LT/GM1. In addition, the same approach can be used to generate other mutants that have reduced affinity for the GM1 receptor (e.g., Ala95Asp). Such mutants are expected to have adjuvanting activity and to exhibit a reduced toxicity profile compared to the wild type toxin.

### Example 22

### Mutant Toxins with High Affinity OS-GM1 Binding in Vitro but Lack GM1 Ganglioside Receptor Binding on Cells

Although the GM1 ELISA is a rapid method for identification of substitutions that perturb GM1 ganglioside or OS-GM1 binding, this screening method does not always predict stable binding to receptors on the cell surface of intact cells. Therefore, mutant or variant toxins should also be assessed for binding to GM1 ganglioside on the surface of a mammalian cell line known to be sensitive to the toxin. An example is the substitution of His in position 57 with Ala (His57Ala) on the B-subunit. This CT mutant exhibits high affinity for OS-GM1 as determined by an ELISA method; however, it is completely inactive when applied to polarized human T84 cells (Rodighiero et al., 2001). In this instance, the His57Ala substitution destabilizes the binding pocket so that at physiological temperature (37° C.) this mutant toxin has low affinity for GM1 ganglioside and fails to crosslink cell surface receptors. As a result, the His57Ala toxin does not induce endosome formation and, therefore, transport of the mutant toxin to the Golgi apparatus and endoplasmic reticulum, where the A subunit proenzyme is activated. Structural analysis shows that His57 forms a weak ionic interaction with the terminal galactose of OS-GM1. Substitution of His57 with Ala is sufficient to cause a shift in the orientation of Glu51 within the pocket, and Ile58 outside the pocket, resulting in an unstable complex with the receptor (Rodighiero et al., 2001). Therefore, variant toxins such as CT-His57Ala are expected to exhibit little or no in vitro and in vivo toxicity, while retaining immune stimulating activity.

Therefore, included in these specifications is the added requirement that mutant or modified AB5 toxins also be evaluated for receptor binding using physiological conditions. Mutant or modified AB5 toxins should be tested for GM1 ganglioside binding using living cells (e.g., Y1, Caco2, CHO and HT29) to establish binding. Mutant toxins that exhibit high or reduced affinity for GM1 gangalioside using an ELISA method of evaluation may not bind to the natural receptor on cultured cells or in vivo.

The ability of mutant or chemically modified toxins to bind to the GM1 ganglioside receptor may be different with different routes of administration. For example, mutant toxins exhibiting poor or no receptor binding at a reduced pH may have no toxicity when administered peroral or topically where the local pH is acidic. In contrast, if the toxin is administered by parenteral injection or nasally, where the local pH is neutral, toxicity may be evident. Therefore, substitutions or other modifications that create toxins unable to form a complex with the natural high affinity receptor under physiological conditions are expected to have reduced or no toxicity in vivo and to be immune stimulating. For these reasons, substitutions that destabilize the binding pocket may exhibit different toxicities when administered by different routes. For the purpose of this disclosure, amino acid substitutions such as the B subunit His57Ala are contemplated. Furthermore, we contemplate that a selective route of administration is specifically selected for the purposes of administering the adjuvant to prevent or avoid binding to high affinity GM1 receptors to avoid local or systemic toxicity without affecting immune stimulating activity.

### Example 23

### Toxin Mutations Outside the OS-GM1 Receptor-Binding Domain

Receptor binding may also be disrupted by introducing mutations outside of the binding pocket. Gly33 is an example of an amino acid outside of the OS-GM1 binding pocket, which is essential to receptor binding. Amino acid substitutions at position 33 that are negatively charged or hydrophobic (Glu, Asp, Ile, Val and Leu) markedly reduces the affinity for OS-GM1, whereas positively charged substitutions (Ala, Lys, Arg) are not destabilizing to ligand binding. Although Gly33 is outside of the binding pocket, the orientation of essential residues within the pocket may slightly shift out of position. For example, the precise position of Tyr12 is important for contact with sialic acid of OS-GM1 and for forming hydrogen bonds between sialic acid and amino acids Glu11 and His13. Amino acid substitutions at position 33, therefore, affect GM1 ganglioside binding by compromising the stability attained by Tyr12. Therefore, included within this specification are mutant AB5 toxins with negatively charged or hydrophobic substitutions at position 33 in the B subunit including, for example, Gly33Glu, Gly33Ile, Gly33Val and Gly33Leu. Mutant toxins with these substitutions are expected to have reduced in vitro and in vivo toxicity and to exhibit immunostimulating activity when co-administered in vivo with a bystander antigen. Not all amino acid substitutions outside the binding pocket affect binding to cell surface GM1 ganglioside. For example, substitution of Glu36 with Gln (Glu36Gln) or Glu51 with Lys (Glu51Lys), do not affect toxin-receptor binding affinity (Jobling and Holmes, 2002).

### Example 24

### Detoxification of LT and CT by Chemical Modification

Another approach to the generation of LT and CT that do not complex with the native receptor is by chemical modification of amino acids essential to binding. There are a number of ways to modify amino acid side chains to affect electrostatic charge and hydrophobicity. For example, the B subunit-binding pocket contains a single tryptophan residue at position 88 (Trp88), which is essential to receptor binding. Trp88 can be modified by the method described by De Wolf et al. (1981). Modification of Trp88 with 2-nitrophenylsulfenyl chloride or 2,4-dinitrophenylsulfenyl chloride (NPS) has been shown to completely prevent the binding of NPS-modified toxins to GM1 ganglioside incorporated into liposomes or expressed on plasma membranes. The chemical reaction can be controlled to cause modification of only Trp88 in each B subunit (5 NPS moieties per holotoxin) without causing modification to Trp residues in the A subunit, as judged by adenylate cyclase activity when erythrocyte or thyroid membranes were treated with nitrophenylsulfenylated (NSP)-toxin. Furthermore, unlike native CT, NPS-CT was shown not to elicit an inflammatory response when injected into rabbit skin (De Wolf et al., 1981). Trp88 may also be selectively modified by formylation. Treatment of toxins with HCl-saturated formic acid results in formylation of Trp88 without causing modification to other amino acids in the binding pocket. Formylated-Trp88 toxins have been shown to lack binding to GM1 ganglioside (Ludwig et al., 1985). Therefore, selective chemical modification of residues involved with toxin binding to the high affinity GM1 ganglioside receptor is an effective way to generate detoxified enterotoxins with biological properties similar to LTGly33Asp and LT/GM1.

The OS-GM1 moiety is stabilized in the binding pocket by a Lys at position 91. Positively charged, Lys91 forms two ionic bonds with sialic acid of OS-GM1. Lys91 can be chemically modified by reacting LT or CT with citraconic anhydride in 0.2 M borate buffer (pH 8). Acylation with acetic anhydride or succinylation with succinic anhydride will neutralize or negatively charge Lys91 (Tsuji et al., 1985). In each case, modifications that affect Lys91 net charge interferes with toxin binding to the negatively charged sialic acid and will prevent the OS-GM1 moiety from entering the receptor pocket (Ludwig et al., 1985). Therefore, chemical modifications to LT or CT that effect the formation of a stable complex with GM1 ganglioside are expected to exhibit a complete or partial reduction in toxicity, although immune stimulating properties are expected to be similar to non-modified toxin.

Two half-cysteines are conserved at amino acid positions 9 and 86. These residues form a single intra-chain disulfide bond in each of the B subunits. Disulfide bond formation is essential to B-pentamer and AB5 holotoxin assembly and to receptor binding. Disulfide bonds can be disrupted by denaturing the toxin with 8 M urea at pH 8.1 and reducing the Cys9 and Cys86 disulfide bond with a reducing agent like dithiothreitol (100 mol/mol Cys). The reformation of the disulfide bonds is prevented by treating the partly denatured toxin with an excess of iodacetamide (2.5 molar excess over dithiothreitol). The reduced toxin is then re-natured by stepped decreases in urea (4M, 2M, 1M and 0.5M) followed by exchange into 0.1 M phosphate buffer (pH 7.5). LT or CT treated by this method does not combine with the high affinity receptor (Ludwig et al., 1985). This approach may be used to generate EtxB and CtxB that do not combine with the natural receptor.

### Example 25

### Toxin Receptor Antagonists

Ultrastructural analysis, site-specific mutagenesis and chemical modification provide insight into the OS-GM1 binding pocket of AB5 toxins. Using this information, combinatorial chemistry can be used to develop small molecules that function as receptor antagonists. In this application, the goal is to design small molecules with high affinity for the receptor-binding pocket within the B subunit. Structure based analysis of how OS-GM1 fits within the binding pocket, provides valuable insight into the design of small molecules, which fit with high affinity into the pocket and thereby block toxin binding to GM1 ganglioside receptors in vivo, much the same way that soluble GM1 ganglioside was used to interfere with the binding of LT or CT to cells. Using galactose, lactose, or Gal-β1,3-GalNAc-β1,4-(NeuAc-α2,3)-Gal-β1,4-Glc-β1 (OS-GM1) as structural backbones for design, new compounds may be synthesized using combinatorial chemistry or, alternatively, existing chemical libraries may be “cherry picked” to select compounds with structural features predicted to fit the receptor binding pocket. For example, existing chemical libraries are evaluated and those compounds with core structures similar to galactose, lactose or OS-GM1 are selected. Substances with the desired core structures, but with different R-groups, are selected for inclusion in the initial screening. Available Chemicals Directory (Molecular Design Ltd., San Leandro, Calif.) is an example of a library, which can be used to identify compounds for screening (Minke et al., 1999a). Alternatively, chemical linkers may be added to galactose, lactose or OS-GM1 core structures. To simplify synthesis, different classes of side groups are covalently coupled to each of the backbone structures from which families of new compounds are synthesized. Gal-βNHCO—(CH₂)ₙ—R and Gal-α-O—(CH₂)₂—NHCO—(CH₂)ₙ—R are examples of linkers that can be covalently coupled to a galactose core structure (Minke et al., 1999b).

Regardless of how the chemical libraries are generated, a rapid screening method is required in order to identify lead compounds. The GM1 ELISA is an ideal assay of screening hundreds or thousands of compounds. The method is essentially as described in the Methods section. Briefly, 96 well microtiter plates are coated with OS-GM1, GM1 ganglioside or GD1b (0.2 μg to 1 μg) in 0.5 M bicarbonate buffer overnight at 4° C. The wells are blocked with 100 μl of phosphate buffered saline with 0.1% BSA and 0.05% Tween-20 (PTB). LT or CT (0.2 μg/ml) is pre-incubated with serially diluted (0.5 mM to 5 mM) test compound for 1-2 hr at room temperature. The test samples (100 μl) are added to the wells for 30 minutes at room temperature and unbound toxin removed by washing the plates. Optimally diluted (usually 1:1,000 to 1:5,000) rabbit anti-LT or CT IgG is added to the wells for 1 hr and the plate washed to remove unbound antibodies. Horseradish peroxidase conjugated anti-rabbit IgG diluted with PTB buffer is added to the wells (100 μl) and the wells washed thoroughly before adding the substrate (o-phenylenediamine in citrate buffer). The color is developed for 30 minutes at room temperature and the OD determined at 450 nm with an ELISA plate reader. Compounds found to block the binding of the toxin to OS-GM1, GM1 ganglioside or GD1b are identified and selected for further evaluation. This approach is used to identify classes of compounds that have some affinity for the OS-GM1 pocket of the B-subunit. Receptor antagonists are further characterized for toxicity in cell-based assays. For example, lead compounds would be evaluated as antagonists of LT or CT cytotoxicity using Y1, Caco2 or CHO cells (Cheng et al., 2000, Giannelli et al., 1997 and Sixma et al., 1992). Those compounds found to prevent in vitro cytotoxicity would then be selected for evaluation as toxin antagonists using one or more animal models. Such screening models include, for example, the patent mouse model (Cheng et al., 2000 and Dickenson and Clements, 1995), rabbit ileal loop model (Giannelli et al., 1997) and murine skin inflammation model (Tables 2, 3). For in vivo testing, LT or CT are admixed with the putative antagonist at different molar ratios prior to administration. The mixture is administered by gavage for the patent mouse model, injection into ileal loops for the rabbit model, or cutaneous injection for the murine model. The amount of enterotoxicity or skin reactogenicity is compared to neat LT or CT to determine the effect of the antagonist on toxicity.

Using this approach, compounds with increased affinity (100 times) over galactose have been identified, although these compounds bind 50,000 times less well than OS-GM1 (Minke et al., 1999a). Through multiple cycles of screening and synthesis, lead compounds are identified. Compounds found to have potential as receptor antagonists include, for example, m-nitrophenyl α-galactoside, p-aminophenyl α-galactoside and melibionic acid. Further modifications to lead compounds can be made to create novel families of compounds with greater receptor affinity, improved pharmacological properties and reduced in vivo toxicity. In addition, it is desirable to select R-groups that do not cause molecular modifications to proteins (e.g., oxidation or deamidation), which could affect immune stimulating activity and shelf stability of the toxin or the vaccine antigen. Therefore, using traditional combinatorial chemistry, new high affinity receptor antagonists can be generated.

Using GM1 ganglioside or OS-GM1 as examples of high affinity receptor antagonists, we have demonstrated the toxicities of LT and CT are attenuated without affecting immune stimulating activity. Likewise, toxins formulated as a complex with other small molecule antagonists can be used and are expected to have the same in vivo properties as toxins pre-adsorbed with GM1 ganglioside or OS-GM1. Although the affinity of compounds like m-nitrophenyl α-galactoside (IC₅₀=0.6-0.7 mM), p-aminophenyl α-galactoside (IC₅₀=4.8-12 mM) and melibionic acid (IC₅₀=5-11 mM) are 100 times greater than D-galactose and lactose (IC₅₀=45-60 mM), the affinity will need to be substantially improved in order to be an effective antagonist in vivo. Compounds with binding affinity in the range 10⁻⁵ M to 10⁻¹⁰ M are desirable for this application. Compounds with affinities equal or greater than GM1 ganglioside (10⁻⁸ M to 10⁻¹⁰ M) are most preferred for this application.

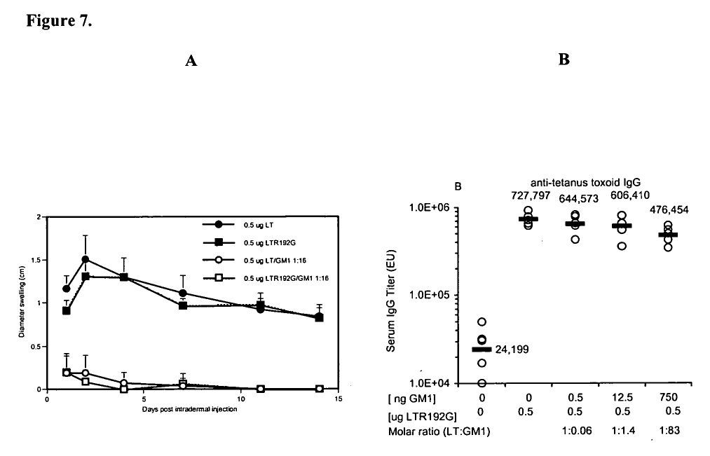

The molar ratio of toxin to antagonist is dependant upon the binding affinity of the antagonist and may be affected by the intended route of administration. As a minimum requirement, the toxin would be formulated by mixing with the antagonist at a ratio where 1, 2, 3, 4 or 5 B-subunits are occupied by one antagonist molecule. With very high affinity antagonists, occupancy of 1 to 4 of the binding pockets is expected to partially destabilize receptor binding on cells (Table 1 and FIG. 7). As we described in Table 1, the LT to GM1 ganglioside ratio of 1:3 was sufficient to cause partial reduction in enterotoxicity in the murine model. Such toxin/antagonist formulations are expected to exhibit a reduced or no toxicity compared to neat toxins. At these molar ratios, toxins are expected to exhibit a reduced affinity for GM1 ganglioside receptor on the cell surface and to exhibit reduced or no cytotoxicity against cultured Y1 cells, for example (Table 5). Since occupancy of the binding pocket is dynamic in solution, or when administered into the body, it is more desirable to have an excess of the antagonist relative to AB5 toxin. For example, high affinity antagonists (Kd=6-7×10⁻⁹ M to 1×10⁻¹⁰ M) such as soluble OS-GM1 or GM1 ganglioside were combined with holotoxins at a molar ratio of 1:15 to 1:30 (toxin to antagonist). This ratio was found to be sufficient to reduce in vivo toxicity and immunogenicity of LT yet did not interfere with potentiating immune responses to bystander antigens (FIGS. 1 and 7). Increasing the molar ratio to 1:30 completely eliminated in vivo toxicity and reduced toxin immunogenicity without affecting the adjuvant activity of the toxin. For antagonists with high affinity for the binding pocket the ideal molar ratio is 1:30. For antagonist with low affinity (i.e., 6.0×10⁻² M to 6.0×10⁻⁴ M) for the B subunit receptor-binding pocket, the antagonist will need to be 1-5×10⁴ times in excess of the toxin.

For purposes of formulation, LT or CT is mixed at a molar ratio that has been determined to be optimal for detoxification without affecting immune stimulating activity. Since LT and CT are stable at refrigerated temperatures in phosphate buffered saline (PBS), the toxin may be formulated with the antagonist and stored at 2-8° C. Alternatively, LT or CT may be mixed with the antagonist and lyophilized or freeze-dried in a pharmaceutical formulation. For use, the dried power is reconstituted in saline or sterile water.

Formulation of the detoxified adjuvant with a vaccine is dependant upon the stability and compatibility of the vaccine with the adjuvant/antagonist formulation. Adjuvant/antagonist may be pre-mixed with the vaccine and supplied as an adjuvanted vaccine. Alternatively, the adjuvant may be supplied separately in a vial or syringe and mixed with the vaccine immediately before intramuscular, subcutaneous or intradermal injection or topical administration (Table 14). For parenteral injection, toxin/antagonist can be administered over a dose range 0.5 μg to 500 μg of protein. The ideal dose range for parenteral vaccination is 0.5 μg to 150 μg. The preferred dose range for injection is 0.5 μg to 50 μg. For topical skin delivery, the same formulations used to deliver wild type LT or CT may also be used to formulate toxin/antagonist and vaccine antigens.

Another way to antagonize LT binding to GM1 in vivo is by adding excess B subunit to the formulation. The B subunit by itself is non-toxic but also considered to be a poor adjuvant. Excess B subunit will compete with the intact holotoxin for binding to GM1 in vivo reducing the interaction of the holotoxin with the GM1 receptor. Such a formulation is expected to be less toxic but with a similar immunostimulatory capacity as LTGly33Asp or LT-GM1 complex.

### Example 26

### Improvement to the in Vivo Delivery of CT and LT-Adjuvants Using Lipophilic Toxin Antagonists

Penetration enhancers are classes of compounds that facilitate the delivery and penetration of co-administered substances across biological membranes. Penetration enhancers include, for example, surfactants, bile salts, fatty acids, sulfoxides, polyols and monohydric alcohols. Penetration enhancers are typically used to improve the delivery of small molecule drugs for transdermal drug delivery. AB5 toxin antagonists may be designed to aid in formulating AB5 adjuvants with a penetration enhancer. The design of the toxin antagonist takes advantage of the structure of the GM1 ganglioside receptor, in which cell surface exposed OS-GM1 is anchored in the lipophilic plasma membrane by ceramide (Gal-β1,3-GalNAc-β1,4-(NeuAc-α2,3)-Gal-β1,4-Glc-β1-ceramide). Ceramide is a diglyceride composed of steric acid and sphingosine. For example, a galactose containing core structure composed of a mono- (e.g., galactose), di- (e.g., lactose), pentasaccharide (e.g., OS-GM1) or other oligosaccharides is covalently coupled to a mono-, di- or triglyceride to produce the galactoside antagonist. The synthetic galactoside is then formulated with a penetration enhancer such as a surfactant (e.g., sodium laurate, Tween 80 or polysorbate), bile salts (e.g., sodium deoxycholate or glycocholate), fatty acids (e.g., oleic acid, glycerides or caprylic acid), polyols (e.g., propylene glycol, polyethylene glycol, glycerol or propanediol), alcohols (e.g., ethanol or isopropyl alcohol) or liposomes. CT or LT are added to the galactoside charged penetration enhancer and the toxin allowed to bind to the exposed saccharide moiety on the surface of a liposome or micell. Alternatively, CT or LT may be mixed with the synthetic galactoside using an effective molar ratio (Table 1 and Example 25). For high affinity antagonists, the toxin and galactoside are thoroughly mixed for 1 hour at ambient or refrigerated temperatures. For low affinity antagonists, the toxin and galactoside are mixed for 12-24 hours at ambient or refrigerated temperatures. The toxin/galactoside complex is then combined with the penetration enhancer. Since CT and LT toxicity is attenuated when the receptor-binding pocket is occupied, the formulated toxin/antagonist/penetration enhancer is admixed with a bystander vaccine or antigen before administration. In addition, the efficiency of topical delivery of LT or CT may be significantly improved by formulating toxin/antagonist with a penetration enhancer to promote delivery of the adjuvant and bystander antigen into the epidermis.

Use of non-receptor binding mutants and chemically modified LT and CT to further attenuate A subunit toxins. Cell intoxication is mediated through the enzymatic activity of a fragment of the A subunit. Toxins bind to host cell GM1 ganglioside receptors through the B pentamer. CT and LT are internalized into the cell within endosomal vesicles and retrograde transported to the Golgi apparatus as the intact holotoxin. The A-subunit (240 amino acids) dissociates from the B pentamer prior to transport into the ER. It is within the ER that the single disulfide bond (Cys187-Cys199) is reduced and, in the case of LT, an enzymatic cleavage between Arg192 and Met195 takes place activating the pro-enzyme and releasing the A1 (residues 1-192) and A2 (193-240) polypeptides (O'Neal et al., 2004). The A1 is transported to the cytosol where it interacts with ADP-ribosylation factors (ARF). The A1 domain has a globular structure and contains the catalytic site of an enzyme that modifies Gsα on the plasma membrane causing an accumulation of intracellular cAMP, prostaglandin production and intestinal fluid accumulation. Mutagenesis has been the primary approach used to define the catalytic domain in the A subunit and to identify residues within the A1 polypeptide that are responsible for cell intoxication. Within the context of this invention, we envision the development of attenuated toxin adjuvants, which combines one or more A1 polypeptide mutations with a B-pentamer having reduced or no affinity for the GM1 ganglioside receptor. The combination results in a more highly attenuated, non-reactogenic adjuvant.

### Example 27

### Further Attenuation of LT Toxins Resistant to Protease Activation

In vivo toxicity of LT toxins is mediated through intracellular activation of the A-proenzyme. Activation requires enzymatic cleavage and reduction of a single disulfide bond. The trypsin-sensitive cleavage site within the A-subunit (187-CGNSSRTITGDTC-199 loop) (SEQ ID NO: 1) has been demonstrated by substitution of the Arg at position 192 with Gly (Arg192Gly) rendering LT resistant to in vitro trypsin activation (Dickenson and Clements, 1995). LT toxicity may be partially attenuated using site directed mutagenesis to substitute the Arg residue at position 192 with Gly in the A subunit to generate LTArg192Gly (International Patent Application WO 96/06627). This substitution renders the LT proenzyme resistant to activation by trypsin digestion. In short term Y1 cell cultures, this mutant LT does not stimulate ADP-ribosyltransferase and cAMP accumulation and lacks enteroxicity when administered peroral to mice (Cheng et al., 2000). This mutant, however, retains mucosal adjuvanting properties (Hagiwar et al., 2001). Although LT-Arg192Gly entrotoxicity has been attenuated, this mutant causes fluid accumulation in the rabbit ileal loop model and it is cytotoxic when cultured with Y1 cells for an extended period (>8 hours) (Giannelli, et al., 1997). Furthermore, intradermal injection of 0.5 μg of LT-Arg192Gly or an equal amount of wild type LT are equally inflammatory and produce induration that persisted for greater than 2 weeks (FIG. 7A). In contrast, when LT-Arg192Gly or LT were pre-adsorbed with soluble GM-1 ganglioside (1:16 molar ratio) before injection, toxin-induced inflammation was completely abolished. These results demonstrate that mutations within the A-subunit of AB5 toxins only partially attenuated in vivo toxicity. LT-Arg192Gly mutant can be rendered completely non-reactogenic and suitable for use as an injected adjuvant provided GM1 ganglioside receptor binding is prevented in vivo. In addition, complete attenuation of LT-Arg192Gly toxicity did not affect its adjuvanting properties since co-administering LT-Arg192Gly/GM1 with tetanus toxoid (TT) elicited a 27-fold increase in anti-TT IgG titers compared to immunization with non-adjuvanted TT (FIG. 7B). LT-Arg192Gly/GM1 potency was equal to non-attenuated wild type LT. Therefore, the toxicity of partially attenuated AB5 toxins can be further attenuated by blocking in vivo binding to high affinity GM1 ganglioside receptors. In this example, we demonstrated that soluble GM1 ganglioside can be used as high affinity receptor antagonist which effectively blocks wild type or partially attenuated AB5 toxins from recognizing GM1 ganglioside receptors in vivo.

An additional way to further attenuate the toxicity of A-subunit mutant toxins, such as LTArg192Gly, is to generate a double mutant toxin. For example, site directed mutagenesis can be used to produce double mutant LT or CT with the A subunit substitution, Arg192Gly, combined with B subunit substitution, Gly33Asp to generate a highly attenuated double mutant. As illustrated in FIG. 7, AB5 toxins with a combination of resistance to enzymatic activation and blocking high affinity receptor binding have a safety profile that is superior to the single A-subunit substitution, without compromising the immune stimulating activity. LT-Arg192Gly/Gly33Asp is a novel composition. A highly attenuated CT adjuvant can be generated using the same strategy.

Alternative methods may also be used to further reduce the toxicity of enzyme resistant mutants like LTArg192Gly. Mutant toxins constructed to be resistant to proenzyme activation may also be chemically modified within the receptor-binding pocket to prevent GM1 ganglioside receptor binding using the methods described in Example 24. Yet another way to further attenuate LTArg192Gly and similar mutants is to block the receptor-binding pocket with a high affinity antagonist as was illustrated in FIG. 7 and Example 25. OS-GM1, p-aminophenyl α-galactoside and other synthetic galactosides are examples of such antagonists.

### Example 28

### A Subunit Mutants with Amino Acid Substitutions Designed to Prevent ADP-Ribosyltransferase Activity

Mutagenesis has been used extensively to generate LT and CT variants with reduced or no ADP-ribosyltransferase activity in vitro. The catalyase activity resides in globular A1 polypeptide. A number of reports have described the effects of various amino acid substitutions on catalytic activity and in vitro and in vivo toxicity. In general, site directed mutagenesis is used to generate amino acid substitutions at various locations within the A1 polypeptide. ADP ribosylating activity is commonly determined by measuring cAMP accumulation in Caco2 cells or morphological changes to Y1, CHO or HT29 cells (reviewed in Spangler, 1992). Enterotoxicity is commonly determined by feeding adult BALB/c mice 1-250 μg of the toxin, harvesting intestines after several hours and determining water accumulation by weight and calculating the gut to carcass ratio (Cheng et al., 2000). Alternatively, the enterotoxicity of mutant and wild type toxins can be determined by injection into isolated ileal loops of rabbits and fluid accumulation determined (Giannelli et al., 1997). As an example, substitution of the Ser in position 63 with Lys (Ser63Lys) in the A subunit of LT, results in a complete loss of ADP ribosylating activity and enterotoxicity (Giannelli et al., 1997 and Stevens et al., 1999). Substitutions of LT Ala72 with Arg (LT-Ala72Arg) (Neidleman et al., 2000) or the CT substitution of Pro106 with Ser (CT-Pro106Ser) exhibit reduced ADP-ribosylating activity in the Y1 cell assay and each mutant has reduced toxicity compared to the wild type toxins, as judged by water accumulation in the rabbit ileal loop model. These mutant toxins, however, potentiate immune responses to a co-administered bystander antigen (e.g., OVA, KLH and Bordetella pertussis) (Pizza et al., 2001), when administered by the nasal or oral routes. In general, higher doses of these A1 mutant holotoxins are required to achieve the same level of adjuvanting activity as the wild type toxins. Because these mutant toxins generally have a residual level of toxicities, and higher doses are usually required to achieve the same adjuvanting response as wild type, many A1 mutant toxins will require additional attenuation before they will be suitable for use as adjuvants in humans and other animals.

A large number of other amino acid substitutions have been made with the A1 polypeptide, which affects catalyase activity of LT. A few examples include substitution of Glu112 with Lys (Glu112Lys), substitution of Ser61 with Phe (Ser61Phe), Ala69 substituted with Gly (Ala69Gly) or His44 substituted with Arg (His44Arg) all lack or have reduced, ADP-ribosyltransferase activity and exhibit reduced cytotoxicity on cultured Y1 (Cheng et al., 2000). In the patent mouse model, Ala69Gly mutant exhibits 50% reduced entertoxicity compared to wild type LT, while the Glu112Gly and Ser61Phe mutants did not elicit fluid accumulation in the intestines of challenged mice (Cheng et al., 2000 and Verniej et al., 1998). The His44Arg mutant LT is less toxic in the Y1 cell assay and in the ileal loop model compared to wild type LT (Hagiwar et al., 2001, Douce et al., 1998 and Douce et al., 1999). Although the cellular and enterotoxicity of these mutants is partly attenuated, they maintained some adjuvanting activity when administered nasally with tetanus toxoid (Cheng et al., 2000).

Other substitutions have been made within and adjacent to the catalytic site in the A1 subunit of LT. Val53Asp and Arg7Lys are within the catalytic site while Val97Lys and Tyr104Lys are substitutions that have been made adjacent to or outside of the catalytic site. In vitro, all of these mutants were found to assemble into LT holotoxins, to be nontoxic to Y1 cells, and to lack ADP-ribosyltransferase activity (Stevens et al., 1999). These include the Ala substituted with Arg at position 72 in the A subunit (LT-Ala72Arg), substitution of Thr50 with Gly or Phe at position 50 in the A subunit (LT-Thr50Gly and (Thr50Pro) and substitution of Val53 substituted with Gly or Pro at position 53 in the A subunit (Val53Gly) and (Val53Pro) (Neidleman et al., 2000 and Verweij et al., 1998). The combination of Gly33Asp B subunit with one or more A subunit catalytic site mutant(s) is expected to further reduce or eliminate in vitro and in vivo toxicity. Likewise, using the methods described above to disrupt GM1 ganglioside binding will results in highly attenuated toxins that can be safely administered as immune stimulating agents to humans and other animals.

In summary, we contemplate that many AB5 toxins with substitutions affecting ADP-ribosyltransferase activity will require further attenuation. Novel highly attenuated adjuvants are generated by constructing toxins with one or more substitution with in the catalytic domain of the A1 polypeptide, which reduces or eliminate ADP-ribosylating activity together with one or more B-subunit substitutions that interfere with GM1 ganglioside binding. Multiple substitutions can readily be made within the LT or CT A and B genes using site directed mutagenesis to introduce base changes that will result in expression of one or more mutations affecting ADP-ribosyltransferase activity and perturb the receptor binding pocket of the B subunit. Double mutant toxins are expected to have little or no toxicity when used at high doses, yet, stimulate antibody and cellular immune responses to co-administered antigens. Amino acid substitutions affecting catalyase activity in the A1 polypeptide combined with the B subunit substitution Gly33Asp are each highly attenuated AB5 adjuvants and represent novel compositions of matter. These include, for example, LT-Ser63Lys/Gly33Asp, LT-Ala72Arg/Gly33Asp, LT-Glu112Lys/Gly33Asp, LT-Ser61Phe/Gly33Asp, LT-Ala69Gly/Gly33Asp, LT-Ser61Phe/Gly33Asp, Ala69Gly/Gly33Asp, LT-His44Arg/Gly33Asp, LT-Val53Asp/Gly33Asp, LT-Arg7Lys/Gly33Asp, LT-Val97Lys/Gly33Asp, LT-Tyr104Lys/Gly33Asp, LT-Thr50Gly/Gly33Asp, LT-Val53Gly/Gly33Asp and CT-Pro106Ser/Gly33Asp. Alternatively, it is also possible to further attenuate the toxicity of toxins defective for ADP-ribosylating activity by blocking GM1 receptor binding by making chemical modifications to essential amino acids required for GM1 ganglioside binding (Example 24) or by complexing these mutant toxins with a receptor antagonist to perturb in vivo receptor binding (Example 25).

### Example 29

### Attenuation by AB5 Toxin Fusion Proteins

Another approach that can be used to partially or fully attenuate the toxicity of CT and LT is to fuse a peptide to the N terminus of the A subunit. Sanchez et al. (2002) demonstrated that it is feasible to express CT holotoxin with heat stable entertoxin (STa) from enterotoxigenic E. coli genetically fused to the N terminus of the A subunit. CTA fusions consisting of APRPGP- (6 mer), (SEQ ID NO: 2) ASRCAELCCNPACPAP- (16 mer) (SEQ ID NO: 3) and ANSSNYCCELCCNPACTGCYPGP- (23 mer) (SEQ ID NO: 4) were constructed and demonstrated to assemble with the B-pentamer to form the holotoxin. The fused holotoxins were shown to have CT activities with the exception of entertoxicity, which was 10 fold reduced in the 6-mer fusion, 100 fold reduced in the 16-mer fusion and 1000 fold reduced in the 23-mer fusion CT compared to wild type CT. The reduced toxicity was attributed to steric interference with the ADP-ribosylating active site in CTA. Furthermore, CT-fusions were shown to retain immuno-potentiating activity when they were co-administered nasally with a bystander antigen (Sanchez et al., 2002).

A similar strategy is to fuse poly-histidine to the N terminus of the LT A-subunit to generate LTA(His₁₀) (De Hann et al., 1999). Although holotoxins were not generated in this study, the fusion protein was shown to have reduced ADP-ribosylation activity in vitro compared to wild type LT. When administered nasally, LTA(His₁₀) failed to elicit serum or mucosal antibodies yet potentiated immune responses against a co-administered antigen (influenza strain B/Harbin/7/94). In the light of the results reported by Sanchez et al. (2002) and the demonstration that LTA(His₁₀) has adjuvanting activity in vivo with reduced toxicity, it is likely that LT holotoxin fusion proteins can also be generated. Blocking or disrupting the ability of CT- and LT-fusion proteins from forming complexes with the GM1 ganglioside receptor is likely to further attenuate the in vivo toxicity of this class of adjuvant.

### Example 30

### Activation of the Skin's Immune Cells by Physical, Mechanical or Chemical Disruption

Although the topical use of adjuvants is an effective and efficient way to stimulate skin dendritic cell activation and to potentiate immune responses to topical administered or injected antigens, dendritic cell activation may also be achieved through other methods, including physical disruption of the stratum corneum and superficial layers of the epidermis. Although it wold not be obvious that trauma to the skin would be useful, in the context of controlled skin injury, with subsequent Langerhans cell activation, skin trauma may be used to enhance the immune response to antigens delivered to the skin. Therefore, as a part of these specifications, we include the use of mechanical, physical and chemical methods to non-specifically activate immune cells in the skin with the intent of promoting immune responses to parenterally injected or topically administered vaccine antigen(s) or antigens delivered to the skin. More specifically, the stimulus is intended to cause the activation of Langerhans cells resident in the epidermis, dendritic cells resident in the dermis or the recruitment of dendritic cells from the blood into treated skin. Immune cell activation may be assessed, for example, by an increase in the expression of co-stimulatory antigens (e.g., MHC class II, CD80 and CD86), endopinocytosis of antigen, morphological changes and migration of dendritic cells from the skin to tissue draining lymph nodes. Ultimately, the effectiveness of the activating stimulus is the potentiation of immune responses to an administered antigen.

The following are examples of how a physical, mechanical or chemical stimulus may be applied to the skin to potentiate the immune response to vaccine antigens. For the purpose of illustration, a mild abrasive was used to disrupt the stratum corneum immediately before a vaccine was administered. In general, the activation stimulus may be applied 1-2 hours before or after the antigen(s) is administered. Ideally, the activating stimulus is simultaneously administered with the vaccine. In the case of injected vaccines, the activating stimulus is most effective when applied directly over or adjacent the site of an intradermal, subcutaneous or intramuscular injection. There may be a specific advantage to targeting the same draining lymph node i.e. to disrupt the skin and deliver the antigen such that these occur in the same draining lymph node field. In the case of topically administered vaccines, the skin is pretreated with the stimulus immediately before or simultaneous with application of the vaccine to the skin.

Improved Immune Response to an Injected Antigen(s) by Mild Disruption of the Stratum Corneum with an Abrasive Pad

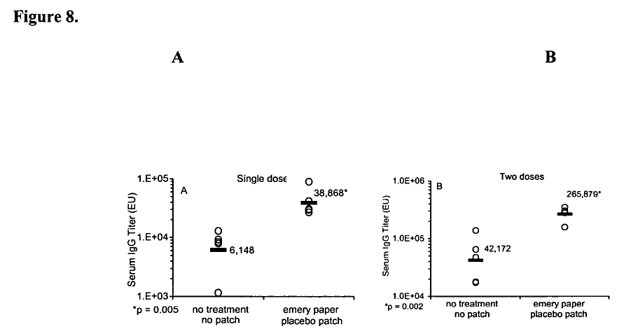

The results in FIG. 8 illustrate the use of a mild abrasive pad (emery paper) as a way to activate skin dendritic cells over the site of immunization. In this example, mice were shaved on the dorsal caudal surface 1-2 days before immunization. Immediately before immunization, half of the mice were pretreated by gently rubbing the shaven skin with a fine grain emery paper (10 strokes) immediately followed by intradermal injection of 0.5 Lf of tetanus toxoid (TT) into the abraded skin. A placebo patch was applied over the injection site overnight. The remaining mice were immunized by intradermal injection of 0.5 Lf of TT without treating the skin. Two weeks following immunization, serum was collected and antibody titers to TT were determined by the ELISA method. As seen in FIG. 8A, animals immunized with one dose of the vaccine without abrading the skin generated relatively low titer anti-TT antibodies (GMT=6,000). In contrast, animals pretreated with the abrasive pad generated significantly higher (p=0.005) anti-TT antibody titers (GMT=38,900). Likewise, after a second dose (FIG. 8B) mice pretreated with emery paper generated significantly higher (p=0.002) anti-TT titers (GMT=266,000) compared to the group that was not pretreated with the abrasive pad (GMT=42,000). These results demonstrate that the immune response to an injected vaccine can be significantly improved by activation of skin dendritic cells with mild abrasion to the skin over the injection site. This technique can be used to potentiate the immune response to a priming dose of vaccine as well as with a booster immunization. The same effects were obtained when vaccines were administered by the subcutaneous and intramuscular routes.

The Immune Response to a Parenteral Injected Antigen is Potentiated by Topical Administration of an Adjuvant to Skin Pretreated with an Activating Stimulus

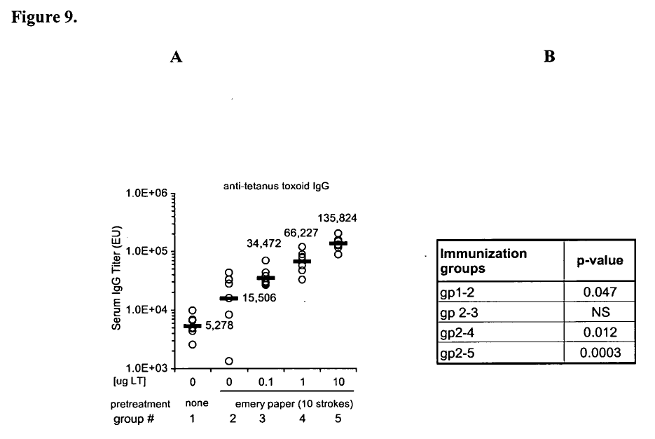

Another way to improve the immune response to an injected antigen(s) is to combine physical disruption of the stratum corneum with topical administration of an adjuvant. To illustrate this example, groups of mice were shaved two days before immunization. Immediately before immunization, the shaven skin was pretreated with emery paper (10 strokes) to disrupt the stratum corneum. A separate group received no skin pretreatment. Groups of 7-8 mice were then immunized by intradermal injection of 0.5 Lf of TT in the pretreated skin. A 1.0 cm² gauze pad affixed to an adhesive backing was loaded with phosphate buffered saline (no LT-adjuvant) or increasing amounts of LT (0.1 μg, 1.0 μg and 10 μg). Patches were removed the next day and the skin rinsed with water. Blood samples were collected two weeks after immunization and serum antibody titers to TT were determined by the ELISA method. The results in FIG. 9 show that the group immunized by intradermal injection with TT without skin pretreatment or adjuvant generated relatively low titer antibodies to TT (GMT=5,300). In contrast, the group treated with the abrasive (no adjuvant) pad generated significantly higher (p=0.047) anti-TT titers (GMT=15,500), again demonstrating that mild trauma to the skin is sufficient to potentiate the immune response to an injected vaccine. The anti-TT titers were further augmented by topical application of LT-adjuvant to skin that was pretreated with the abrasive pad. As seen in FIG. 9, anti-TT titers were significantly increased (p is less than or equal to 0.012) by topical application of 1 or 10 μg of LT to pretreated skin. These results indicate that an activating stimulus (mild abrasion) over the site of parenteral injection combined with topical application of an adjuvant (e.g., LT) significantly potentiates the immune response to an injected vaccine. The same effect was obtained when the vaccine was administered by subcutaneous or intramuscular routes.

Mild Disruption of the Outer Layers of the Skin Potentiates the Immune Response to Topically Administered Antigens

The results in table 15 illustrate the effect of disrupting the stratum corneum with an abrasive upon the generation of an immune response to a topically administered vaccine. In this example, mice were shaved dorsal caudal 1-2 days before immunization. Immediately before topical immunization, the shaven skin was hydrated and mildly pretreated with emery paper to disrupt the stratum corneum. Then 50 μl PBS containing 10 Lf of TT with and without LT adjuvant was applied to the skin for 1 h after which the skin was rinsed with lukewarm tap water. Groups of 10 mice were immunized twice (study days 1 and 15) and serum anti-TT IgG titers were determined by an ELISA method. As is evident from examination of Table 15, the group topically immunized with TT alone without use of a skin abrasive had a very poor immune response (GMT=39). In contrast, the group pretreated with an abrasive pad and received TT generated high antibody titers to TT (GMT=40,725). Furthermore, the group topically immunized with LT-adjuvanted TT generated even higher titer antibodies to TT (GMT=193,986). These results clearly demonstrate that the immune response to a topically administered antigen(s) is significantly improved by disrupting the stratum corneum immediately before topically administering the vaccine. In this example, the improved immune response is due in part to the disruption of the stratum corneum and improving the delivery of the vaccine to resident dendritic cells and in part due to a non-specific activation of dendritic cells elicited by abrading the skin. As demonstrated in this example, the combination of skin pretreatment and co-administering an adjuvant with the antigen is a highly effective way to stimulate immune responses to the bystander antigen.

TABLE 15
Serum IgG to Tetanus Toxoid (TT) after transcutaneous immunization (TCI)
with and without skin abrasion

| | | | | | Assay values 1-10 | | | | | |
|---|---|---|---|---|---|---|---|---|---|---|
| | | | | | (Elisa Units) | | | | | |
| | | | | | Individual Mice | | | | | |
| | antigen | adjuvant | | | 1 | 2 | 3 | 4 | 5 | Geometric |
| Groups | (Lf) | (μg) | pretreatment | detecting | 6 | 7 | 8 | 9 | 10 | mean |
| 1 | TT (10) | — | hydration | TT IgG | 21 | 27 | 13 | 1119 | 119 | 39 |
| | | | | | 46 | 17 | 43 | 7 | nsa | |
| 2 | TT (10) | — | sandpaper | TT IgG | 38607 | 5484 | 19316 | 73699 | 27961 | 40725 |
| | | | | | 66563 | 91558 | 87180 | 45328 | 61830 | |
| 3 | TT (10) | LT(10) | sandpaper | TT IgG | 188314 | 222731 | 178753 | 88333 | 192934 | 193986 |
| | | | | | 201494 | 169482 | 379919 | 73992 | 615173 | |

C57B1/6 mice were immunized topically with tetanus toxoid (TT) alone or mixed with LT. The skin was pretreated by hydration only or hydration + 10 strokes of sandpaper. Two weeks after the second immunization, serum samples were collected and the serum antibody titers against TT were determined by ELISA.

When administration is topical, the skin can be treated prior to, simultaneously with, or after, administration of the formulation/formulations. One or more of the following can be used for such treatment: abrasives; micro-dermabraders; devices comprising microprojections; tape-stripping; chemical peels; devices which create microchannels, micropores or both; micro-needle arrays; high frequency ultrasound; thermal ablation or laser ablation. A number of devices and methods may be used to prepare the skin for immunization. The objective is to administer a minimally invasive treatment that disrupts or penetrates the stratum corneum and/or the outermost layers of the epidermis. As described in these examples, abrasives may be used to buff the skin over the site of an injection or to buff the skin before topical administration of the vaccine. Common medical devices used to prepare the skin for electrodes may be used. Examples of such devices include emery paper (GE Medical Systems), ECG Prep Pads (Marquette Medical Systems) and Electrode Prep pads (Professional Disposables, Inc.). Similar to the use of abrasive pads, micro-dermabrasion may also be used to treat the skin before immediately before immunization. In practice, the micro-dermabrader propels aluminum oxide or sodium chloride crystals that strike the skin and produce superficial trauma by removal of the stratum corneum and superficial layers of the skin. Other devices such as OnVax (Beckton Dickinson) may also be used for this application. These devices are designed with up to 400 microprojections/cm² mounted on a hand held applicator which is raked over the skin. The microprojections are 200 to 300 μm long and create furrows through the stratum corneum. The trauma caused by disrupting the stratum corneum and penetrating the epidermis is sufficient to cause trauma and non-specific activation of dentritic cells in the treated area. Another technique commonly used to remove the stratum corneum is by stripping with tape. D-squame tape (CuDerm Corporation) is commonly used for this application although other tape may also be used. It is also possible that devices causing injury to deeper layers of the skin will have similar adjuvanting effects.

Chemical peel is a technique used to treat photoaging. Various agents may be used to remove the outer layers of the skin. For example, alpha-hydroxy acids, trichloroacetic acid (TCA) and phenol are commonly used. Within the context of promoting immune responses to topically administered vaccines, care is required to minimize the interaction between the chemical agent and the vaccine and the adjuvant. In practice, the chemical agent would be applied to the skin for a prescribed period and washed away before application of the vaccine and adjuvant. In the case of parenteral injected vaccines, the vaccine may be injected followed by application of the chemical agent.

Devices designed to aid in percutaneous delivery of small molecule drugs may also be used to cause mild trauma to the skin. Such devices are designed to create microchannels or micropores that penetrate through the stratum corneum and into the dermis where small molecular weight drugs are delivered to the micro-vessels. In the context of skin immunization, these devices can be used to create trauma by disrupting the stratum corneum and penetrating into the epidermis. Several technologies have been developed and can be used to in the context of this example to cause mild trauma in different layers of the skin (stratum corneum, epidermis and dermis) providing activation stimulus to Langerhans cells and dermal dendritic cells. A number of devices have been developed for creating microchannels or micropores in skin. These devices differ in the method used to create the penetrations. For example, micro-needle arrays are designed to have hundreds or thousands of micro-needles in a small area (cm²). The depth of penetration (25 μm to >400 μm) is controlled by shaft length.

Superficial trauma can also be created by the use of high frequency ultrasound and thermal energy and light. Focused ultrasound can be used to cause mechanical and thermal disruption to skin. Sonication at 1-2 W/cm² is used to cause minimal penetration through the stratum corneum, while sonication greater than or equal to 3 W/cm² causes penetrations into the epidermis. The amount of trauma can be controlled by time. In addition, the stratum corneum may be vaporized by laser ablation or by thermal ablation. Regardless of the method used to induce trauma to the skin, the common objective is to induce superficial trauma to the outer layers of the skin resulting in the stimulation of resident immune cells in the skin. With increased trauma to the skin, it is also likely that dendritic cells from the blood are recruited to the traumatized tissue. In this case, blood dendritic cells may also contribute to potentiation of the immune response to an administered vaccine.

### Example 31

### Controlling Dose and Reactogenicity by Superficial Placement of Antigens and Adjuvants in the Skin

Skin dendritic cells are concentrated in the epidermal and dermal layers of the skin. The objectives of skin pretreatment in the context of skin immunization is two fold: 1) to provide access of antigens and adjuvants to immune cells resident in these superficial layers of skin and 2) to stimulate the dendritic cell activation and maturation through either mild trauma to the skin or through delivery of an adjuvant that is immunostimulating. Therefore, pretreatment methods and devices that accurately provide access of the vaccine and adjuvant in the epidermis are most effective ways to reduce the dose of vaccine and adjuvant that is required for producing a maximal immune response. A second benefit to superficial delivery is to reduce or eliminate reactogenicity elicited by the vaccine and adjuvant. In humans, the depth of the stratum corneum may vary between individuals and may vary in different anatomical sites. However, a range of 5 μm to ˜20 μm is commonly reported in the literature. The epidermis is approximately 100 μm to 150 μm and the dermis 1,000 to 4,000 μm. Skin treatment methods that cause disruption of the stratum corneum and epidermis at a depth of about 5 μm to about 150 μm, for example at a depth of 40 μm to about 60 μm, are effective for delivery of large molecular weight antigens and adjuvants to immune cells in the skin. Therefore, laser and thermal devices that vaporize the stratum corneum are useful for delivery of vaccines to denuded epidermal surface (5-20 μm). Ultrasound, thermal filament and micro-needle devices that create microchannels and micropores in the skin can be used to deliver vaccines and adjuvants into the epidermis. In this case, the objective is to limit the depth of penetration to the dermis without entering the epidermis. Penetration to a depth of 25 μm to 100 μm is desirable for epidermal delivery. In some instances, it will be desirable to deliver antigens and adjuvants to dendritic cells resident in the dermal layers of the skin. In this case, creating microchannels and micropores 1,000 to 4,000 μm deep will be required. Ultrasound, thermal filament and micro-needle devices are suitable for creating penetrations to this depth.

In summary, a number of technologies exist for producing penetrations in skin or for removal of different layers of skin. As disclosed here, these various procedures and devices can also be adapted for use with skin immunization. Regardless of the treatment technique or of the device used, the primary intent is to deliver antigens and adjuvants to immune cells in the epidermis and dermis with the intent of stimulating an immune response to the administered antigen.

References

1. Aman, A, Fraxer, S, Merritt, E, Rodigherio, C, Kenny, M, Ahn, M, Hol, W, Williams, N, Lencer, W and Hirst, T. A mutant cholera toxin B subunit that binds GM1-ganglioside but lacks immunomodulatory or toxic activity. Proc. Natl. Acad. Sci. USA. 2001. 98:8536-8541.
2. Ban, E, Dupre, L, Hermann, E, Rohn, W, Vendeville, C, Quatannens, Ricciardi-Castagnoli, P, Cpron, A and Riveau, G. CpG motifs induce Langerhans cell migration in vivo. Intl Immunol. 2000. 12:737-745.
3. Beignon, A, Briand, J, Muller, S and Partidos, C. Immunization onto bare skin with heat-labile enterotoxin of Escherichia coli enhances immune responses to coadministered protein and peptide antigens and protects mice against lethal toxin challenge. Immunol. 2001. 102(3):344-351.
4. Bone, H, Eckholdt, S and Williams, N. Modulation of B lymphocyte signaling by the B subunit of Escherichia coli heat-labile eriterotoxin. International Immunology. 2002. 14(6):647-58.
5. Buerrant, R, Brunton, L, Schnaltman, T, Rebhun, L and Gilman, A. Cyclic adenosinemonophosphate and alteration of Chinese hamster ovary cell morphology: A rapid, sensitive in vitro assay of the enterotoxins of Vibrio cholerae and Escherichia coli. Infect. Immun. 1974. 10:320-327.
6. Chen, T, Lin, C, Loa, C, Chen, T L, Shang, H, Hwang, J and Hui, C. A nontoxic Pseudomonas exotoxin A induces active immunity and passive protective antibody against Pseudomonas exotoxin A intoxication. J Biomed Sci. 1999a. 6(5):357-363.
7. Chen T, Shang, H, Chen, T, Lin C, Hui, C and Hwang, J. Recombinant protein composed of Pseudomonas exotoxin A, outer membrane proteins I and F as vaccine against P. aeruginosa infection. Appl Microbiol Biotechnol. 1999b. 52:524-533.
8. Cheng, E, Cardenas-Freytag, L and Clements, J. The role of cAMP in mucosal adjuvanticity of Escherichia coli heat-labile entertotoxin (LT). Vaccine. 2000. 18:38-49.
9. Clements, J, Sack, D, Harris, J, Chakraborty, J, Neogy, P, Stanton, B, Juda, N, Khan, M, Kay, B, Khan, M. Cross-protection by B subunit-whole cell cholera vaccine against diarrhea associated with heat-labile toxin-producing enterotoxigenic Escherichia coli: Results of a large-scale field trial. J Infect Dis. 1988. 158:372-377.
10. Condaminet, B, Peguet-Navarro, J, Stahl, P, Dalbiez-Gauthier, C, Schmitt, D and Berthier-Vergnes, O. Human epidermal Langerhans cells express the mannose-fucose binding receptor. Eur J Immunol. 1998. 28:3541-3551.
11. Couch, R. Nasal vaccination, Escherichia coli enterotoxin, and Bell's Palsy. N Engl J Med. 2004. 350:860-861.
12. de Haan, L, Holtrop, M, Verweij, W, Agsteribbe, E and Wilschut J. Mucosal immunogenicity of the Escherichia coli heat-labile enterotoxin: Role of the A subunit. Vaccine. 1996. 14(4):260-66.
13. de Haan, L, Verweij, W, Feil, I, Holtrop, M, Hol, W, Agsteribbe, E and Wilschut, J. Role of GM1 binding in the mucosal immunogenicity and adjuvant activity of the Escherichia coli heat-labile enterotoxin and its B subunit. Immunol. 1998. 94:424-430.
14. de Hann, L, Holtrop, M, Verweij, W, Agsteribbe, E and Wilschut, J. Mucosal immunogenicity and adjuvant activity of the recombinant A subunit of the Escherichia coli heat-labile enterotoxin. Immunol. 1999. 97:706-13.
15. De Wolf, M, Fridkin, M, Epstein, M and Kohn, L. Structure-function studies of cholera toxin and its A and B protomers. J Biol Chem. 1981. 256(11):5481-88.
16. Dickinson, B and Clements, J. Dissociation of Escherichia coli heat-labile enterotoxine adjuvanticity from ADP-ribosyltransferase activity. Infect Immun. 1995. 63(5):1617-23.
17. Dong, X, Storkus, W and Salter, R. Binding and uptake of agalactosyl IgG by mannose receptor on macrophages and dendritic cells. J Immunol. 1999. 163:5427-5434.
18. Douce, G, Giuliani, M, Giannelli, V, Pizza, M, Rappuoli, R, Dougan, G. Mucosal immunogenicity of genetically detoxified derivatives of heat labile toxin from Escherichia coli. Vaccine. 1998. 16(11-12):1065-75.
19. Douce, G, Giannelli, V, Pizza, M, Lewis, D, Everest, P, Rappuoli, R and Dougan, G. Genetically detoxified mutants of heat-labile toxin from Escherichia coli are able to act as oral adjuvants. Infect Immun. 1999. 67(9):4400-06.
20. Edelman. An overview of adjuvant use. In: Vaccine Adjuvants (O'Hagan, ed.) Humana Press, Totowa, (2000) pp. 1-29.
21. Elson & Dertzbaugh. Mucosal adjuvants. In: Handbook of Mucosal Immunology (Ogra et al., eds.) Academic Press, San Diego. 1994. pg. 391.
22. Enioutina, E, Visic, D and Daynes, R. The induction of systemic and mucosal immune responses to antigen-adjuvant compositions administered into the skin: alterations in the migratory properties of dendritic cells appears to be important for stimulating mucosal immunity. Vaccine. 2000. 18:2753-2767.
23. Hammond, S, Walwender, D, Alving, C and Glenn, G. Transcutaneous immunization: T-cell responses and boosting of existing immunity. Vaccine. 2001. 19:2701-2707.
24. Hagiwar, Y, Tsuji, T, Iwasaki, T, Kadowaki, S, Asanuma, H, Chen, Z, Komase, K, Suzuki, Y, Effectiveness and safety of mutant Escherichia coli heat-labile enterotoxin (LT H44A) as an adjuvant for nasal influenza vaccine. Vaccine. 2001. 19:2071-79.
25. van Ginkel, F, Jackson, R, Yuki, Y and McGhee, J. The mucosal adjuvant cholera toxin redirects vaccine proteins into olfactory tissues. J Immunol. 2000. 165:4778-4782.
26. Giannelli, V, Fontana, M, Giuliani, M, Guangcai, D, Rappuoli, R and Pizza, M. Protease susceptibility and toxicity of heat-labile enterotoxins with a mutation in the active site or in the protease-sensitive loop. Infect Immun. 1997. 65(1):331-34.
27. Glenn, G, Scharton-Kersten, T, Vassell, R, Mallett, C, Hale, T and Alving, C. Transcutaneous immunization with cholera toxin protects mice against lethal mucosal toxin challenge. J. Immunol. 1998. 161:3211-3214.
28. Glenn, G, Taylor, D, Li, X, Frankel, S, Montemarano, A and Alving, C. Transcutaneous immunization: A human vaccine delivery strategy using a patch. Nat. Med. 2000. 6:1403-1406.
29. Guebre-Xabier, M, Hammond, S, Epperson, D, Yu, J, Ellingsworth, L and Glenn, G. Immunostimulant patch containing heat-labile enterotoxin from Escherichia coli enhances immune responses to injected influenza virus vaccine through activation of skin dendritic cells. J Virol. 2003. 77:5218-5225.
30. Guidry, J, Cardenas, L, Cheng, E and Clements, J. Role of receptor binding in toxicity, immunogenicity, and adjuvanticity of Escherichia coli heat-labile enterotoxin. Infec Immun. 1997. 65(12):4943-4950.
31. Jobling, M and Holmes, R. Mutational analysis of ganglioside GM 1-binding ability, pentamer formation, and epitopes of cholera toxin B (CTB) subunits and CTB/Heat-labile enterotoxin B subunit chimeras. Infect Immun. 2002. 70(3):1260-71.
32. Krueger, K and Barbieri, J. The family of bacterial ADP-ribosylating exotoxins. Clin Microbiol Rev. 1995. 8:34-47.
33. Lian et al. Formulation of HIV-envelope protein with lipid vesicles expressing ganglioside GM1 associated to cholera toxin B enhances mucosal immune responses. Vaccine. 2000. 18:604-611.
34. LiCalsi, C, Maniaci, M, Christensen, T, Phillips, E, Ward, G and Witham, C. A powder formulation of measles vaccine for aerosol delivery. Vaccine. 2001. 19:2629-2636.
35. Ludwig, D, Holmes, R and Schoolnik, G. Chemical and immunochemical studies on the receptor-binding domain of cholera toxin B subunit. J Biol Chem. 1985. 260(23): 12528-34.
36. Lycke, N. Tsuji, T and Holmgren, J. The adjuvant effect of Vibrio cholerae and Escherichia coli heat-labile enterotoxins is linked to their ADP-ribosyltransferase activity. Eur J Immunol. 1992. 22:2277-2281.
37. Merritt, E, Sixma, T, Kalk, K, van Zanten, B and Hol, W. Galactose-binding site in Escherichia coli heat-labile enterotoxin (LT) and cholera toxin (CT). Mol Microbiol. 1994. 13:745-53.
38. Merritt, E, Sarfaty, S, van den Akker, F, L'Hoir, C, Martial, J and Hol W. Crystal structure of cholera toxin B-pentamer bound to receptor GM1 pentasaccharide. Protein Sci. 1994. 3(2):166-75.
39. Merritt, E, Sarfaty, S, Jobling, M, Chang, T, Holmes, R, Hirst, T and Hol, W. Structural studies of receptor binding by cholera toxin mutants. Protein Sci. 1997a. 6:1516-1528.
40. Merritt, E, Sarfaty, S, Feil, I and Hol, W. Structural foundation for the design of receptor antagonists targeting Escherichia coli heat-labile enterotoxin. Structure. 1997b. 5:1458-99.
41. Merritt, E, Kuhn, P, Sarfaty, S, Erbe, J, Holmes, R and Hol, W. The 1.25 A resolution refinement of the cholera-toxin B pentamer: evidence of peptide backbone strain at the receptor-binding site. J Mol Biol. 1998. 282:1043-1059.
42. Minke, W, Roach, C, Hol, W and Verlinde, C. Structure-based exploration of the ganglioside GM1 binding sites of Escherichia coli heat-labile enterotoxin and cholera toxin for the discovery of receptor antagonists. Biochemistry. 1999a. 38:5684-92.
43. Minke, W, Hong, F, Verlinde, C, Hol, W and Fan, E. Using a galactose library for exploration of a novel hydrophobic pocket in the receptor-binding site of the Escherichia coli heat-labile enterotoxin. J Biol Chem. 1999b. 274(47):33469-73.
44. Nashar, T, Webb, H, Eaglestone, S, Williams, N and Hirst, T. Potent immunogenicity of the B subunits of Escherichia coli heat-labile enterotoxin: Receptor binding is essential and induces different modulation of lymphocyte subsets. Proc Natl Acad Sci USA. 1996. 93:226-30.
45. Nashar, T, Hirst, T and Williams, N. Modulation of B-cell activation by the B subunit of Escherichia coli enterotoxin: Receptor interaction up-regulates MHC class II, CD40, CD25 and ICAM-1. Immunology. 1997. 91:572-78.
46. Nashar, T, Betteridge, Z and Mitchell, R. Evidence for a role of ganglioside GM1 in antigen presentation: Binding enhances presentation of Escherichia coli enterotoxin B subunit (EtxB) to CD4⁺ T cells. International Immunology. 2001. 13(4):541-51.
47. Neidleman, J, Vajdy, M, Ugozzoli, M, Ott, G and O'Hagon, D. Genetically detoxified mutants of heat-labile enterotoxin from Escherichia coli are effective adjuvants for induction of cytotoxic T cell responses against HIV-gag-p55. Immunology. 2000. 101:154-60.
48. O'Neal, C, Amaya, E, Jobling, M, Holmes, R and Hol, W. Crystal structures of an intrinsically active cholera toxin. Biochemistry. 2004. 43:3772-82.
49. Pizza, M, Giuliani, M, Fontana, M, Monaci, E, Douce, G, Dougan, G, Mills, K, Rappuoli, R and Del Giudice, G. Mucosal vaccines: Non-toxic derivatives of LT and CT as mucosal adjuvants. Vaccine. 2001. 19:2534-41.
50. Rodighiero, C. Fujinaga, Y, Hirst, T and Lencer, W. A cholera toxin B-subunit variant that binds ganglioside GM1 but fails to induce toxicity. J Biol Chem. 2001. 276(40):36939-45.
51. de la Salle, H, Haegel, -Kronenberger, H, Bauxinger, H, Astier, A, Cazenave, J, Fridman, W, Sautes, C, Teillaud, J, Hanau, D and Bieber, T. Functions of Fc receptors on human dendritic Langerhans cells. Intl Re. Immunol. 1997. 16(1-2):187-203.
52. Sanchez, J, Wallerstrom, G, Fredriksson, M, Anstrom, J, and Holmgren, J. Detoxification of cholera toxin without removal of its immunoadjuanticity by the addition of (STa-related) peptides to the catalytic subunit. J Biol Chem. 2002. 277(36):33369-77.
53. Scharton-Kersten, T, Yu, J, Vassell, R, O'Hagan, D, Alving, C and Glenn, G. Transcutaneous immunization with bacterial ADP-ribosylating exotoxins, subunits, and unrelated adjuvants. Infect Immun. 2000. 68:5306-5313.
54. Sears, C and Kaper, J. Enteric bacterial toxins: Mechanisms of action and linkage to intestinal secretion. Microbiol Rev. 1996. 60:167-215.
55. Seo, N, Tokura, Y, Nishijima, T, Hashizume, H, Furukawa, F and Takigawa, M. Percutaneous peptide immunization via corneum barrier-disrupted murine skin for experimental tumor immunoprophylaxis. Proc Natl Aca. Sci USA. 2000. 97:371-376.
56. Sixma, T, Pronk, S, Kalk, K, Wartna, E, van Zanten, B, Witholt, B and Hol, W. Crystal structure of a cholera toxin-related heat-labile enterotoxin from E. coli. Nature. 1991. 351:371-377.
57. Sixma, T, Pronk, S, Kalk, K, van Zanten, B, Berghuis, A and Hol W. Lactose binding to heat-labile enterotoxin revealed by X-ray crystallography. Nature. 1992. 355:561-64.
58. Snider, D. The mucosal adjuvant activities of ADP-ribosylating bacterial exotoxins. Crit Rev Immunol. 1995. 15:317-384.
59. Spangler, B. Structure and function of cholera toxin and the related Escherichia coli heat-labile enterotoxin. Microbiol Review. 1992. 56:622-47.
60. Stevens, L, Moss, J, Vaughan, M, Pizza, M and Rappuoli, R. Effects of site-directed mutagenesis of Escherichia coli heat-labile enterotoxin on ADP-ribosyltransferase activity and interaction with ADP-ribosylation factors. Infect Immun. 1999. 67(1):259-65.
61. Takamiya, K, Yamamoto, A, Furukawa, K, Yamashiro, S, Shin, M, Okada, M, Fukumoto, S, Haraguchi, M, Takeda, N, Fujimura, K, Sakae, M, Kishikawa, M, Shiku, H, Furukawa, K and Aizawa, S. Mice with disrupted GM2/GD2 synthase gene lack complex gangliosides but exhibit only subtle defects in their nervous system. Proc Natl Acad Sci USA. 1996. 93:10662-10667.
62. Tsuji, T, Honda, T, Miwatani, T, Wakabayashi, S and Masubara, H. Analysis of receptor-binding site in Escherichia coli enterotoxin. J Biol Chem. 1985. 260(14):8552-58.
63. Tamura, S, Yamanaka, A, Shimohara, M, Tomita, T, Komase, K, Tsuda, Y, Suzuki, Y, Nagamine, T, Kawahara, K, Danbara, H. Synergistic action of cholera toxin B subunit (and Escherichia coli heat-labile toxin B subunit) and a trace amount of cholera whole toxin as an adjuvant for nasal influenza vaccine. Vaccine. 1994. 12:419-426.
64. Tomasi, M, Dertzbaugh, M, Hearn, T, Hunter, R and Elson, C. Strong mucosal adjuvanticity of cholera toxin within lipid particles of a new multiple emulsion delivery system for oral immunization. Eur J Immunol. 1997. 27:2720-2725.
65. Truitt, R, Hanke, C, Radke, J, Mueller, R and Barbieri, J. Glycosphingolipids are novel targets for T-cell suppression by the B subunit of recombinant heat-labile enterotoxin. Infect Immun. 1998. 66(4):1299-1308.
66. Udey, M. Cadherins and Langerhans cell immunobiology. Clin Exp Immunol. 1997. 107:6-8.
67. Valladeau, J, Ravel, 0, Dezutter-Dambuyant, C, Moore, K, Kleijmeer, M, Lui, Y, Duvert-Frances, V, Vincent, C, Schmitt, D, Davoust, J, Caux, C, Lebecque, S and Saeland, S. Langerin, a novel c-type lectin specific to Langerhans cells, is an endocytic receptor that induces the formation of Birbeck granules. Immunity. 2000. 12:71-81.
68. Verweij, W, de Haan, L, Hotrop, M, Agsteribbe, E, Brands, R, van Scharrenburg, G and Wilschut. Mucosal immunoadjuant activity of recombinant Escherichia coli heat-labile enterotoxin and its B subunit: Vaccine. 1998. 16(2):2069-76.
69. Williams, N, Stasiuk, L, Nashar, T., Richards, C, Lang, A, Day, M and Hirst, T. Prevention of autoimmune disease due to lymphocyte modulation by the B-subunit of Escherichia coli heat-labile enterotoxin. Proc Natl Acad Sci USA. 1997. 94:5290-95.
70. Yu, J, Cassels, F, Scharton-Kersten, T, Hammond, S, Hartman, A, Angov, E, Corthesy, B, Alving, C and Glenn, G. Transcutaneous immunization using colonization factor and heat-labile enterotoxin induces correlates of protective immunity for enterotoxigenic Escherichia coli. Infect Immun. 2002. 70:1056-1068.
71. Zhang, R, Scott, D, Westbrook, M, Nance, S, Spangler, B, Shipley, G and Westbrook, E. The three-dimensional crystal structure of cholera toxin. J Mol Biol. 1995. 251:563-573.

All references, articles, books, patents, scientific articles and published patent applications cited above are indicative of the level of skill in the art and are incorporated herein in their entirety by reference.

All modifications and substitutions that come within the meaning of the claims and the range of their legal equivalents are to be embraced within their scope. Thus, all possible combinations and permutations of the individual elements disclosed herein are intended to be considered part of the invention. From the foregoing, it would be apparent to a person of skill in this art that the invention can be embodied in other specific forms without departing from its spirit or essential characteristics.

We claim:

1. A method of inducing an antigen-specific immune response to one or more antigens in a subject in need thereof comprising administering a first formulation comprising at least one GM-1 binding deficient exotoxin and a second formulation comprising at least one antigen, in an amount sufficient to induce said antigen-specific immune response in said subject, wherein said administering is selected from the group consisting of intradermal, intramuscular, subcutaneous and topical.

2. The method of claim 1, wherein said first formulation is administered intradermally, intramuscularly or subcutaneously and said second formulation is administered topically.

3. The method of claim 1, wherein both formulations are administered intradermally, intramuscularly or subcutaneously.

4. The method of claim 1, wherein both formulations are administered topically.

5. The method of claim 4, wherein the site of administration of the first formulation is separate from the site of administration of the second formulation.

6. A method of inducing an antigen-specific immune response to one or more antigens in a subject in need thereof comprising administering a formulation comprising at least one GM-1 binding deficient exotoxin and at least one antigen, in an amount sufficient to induce said antigen-specific immune response in said subject, wherein said administering is selected from the group consisting of intradermal, intramuscular, subcutaneous and topical.

7. The method of claim 1 or claim 6, wherein said at least one antigen is an ETEC antigen.

8. A method of inducing an immune response in a subject in need thereof comprising administering at least one GM-1 binding deficient exotoxin in an amount sufficient to induce said immune response in said subject, wherein said administering is selected from the group consisting of intradermal, intramuscular, subcutaneous and topical.

9. The method of claim 6 or 8, wherein the administration is topical.

10. The method of any one of claims 1, 6 or 8, wherein said administration is topical and further comprises treating skin prior to, simultaneously with, or after, said administration.

11. The method of claim 10, wherein said treating comprises treatment with one or more of: abrasives; micro-dermabraders; devices comprising microprojections; tape-stripping; chemical peels; devices which create microchannels, micropores or both; micro-needle arrays; high frequency ultrasound; thermal ablation or laser ablation.

12. The method of claim 11, wherein said treatment disrupts the stratum corneum from about 5 microns to about 150 microns.

13. The method of claim 12, wherein said treatment disrupts the stratum corneum from about 40 microns to about 60 microns.

14. The method of any one of claims 1, 6 and 8, wherein said GM-1 binding deficient exotoxin is produced by substituting one or more amino acids in at least one B subunit of the exotoxin and/or coupling at least one B subunit of the exotoxin to a molecule effective to inhibit binding to GM-1.

15. The method of claim 14, wherein the substitution comprises at least one point mutation in the GM-1 binding pocket.

16. The method of claim 15, wherein the GM-1 binding deficient exotoxin is a modified bacterial ADP-ribosylating exotoxin (bARE).

17. The method of claim 16, wherein said GM-1 binding deficient exotoxin is LTG33D.

18. The method claim 14, wherein the molecule is selected from the group consisting of a ganglioside, a B subunit-binding portion of a ganglioside, a low density lipoprotein receptor-related protein (LRP), a B subunit-binding portion of LRP, an alpha macroglobulin receptor, and a B subunit-binding portion of an alpha macroglobulin receptor.

19. The method of claim 18, wherein said ganglioside is selected from the group consisting of GM1, GM1 mutants, partial GM1 molecules, GM2, GM3, GD2, GD3 and GD1b.

20. The method of claim 18, wherein the ganglioside comprises a sialic acid and a galactose residue.

21. The method of claim 14, wherein said molecule is a ligand selected from the group consisting of mannose, immunoglobulins, CpG, integrin motifs and any combination thereof.

22. The method of claim 18 or 21, wherein the GM-1 binding deficient exotoxin is a modified bacterial ADP-ribosylating exotoxin (bARE).
